SEQUENCE LISTING (43 sequences)

SEQ ID NO: 1 - protein, 462 aa, Artificial Sequence
  HIS TAG AND TEV CLEAVAGE SITE
MHHHHHHDYDIPTTENLYFQGMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 190 aa, Artificial Sequence
  TAU 166 PEPTIDE WITH HIS TAG AND TEV CLEAVAGE SITE
MSYYHHHHHHDYDIPTTENLYFQGEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQ

SEQ ID NO: 9 - protein, 166 aa, Artificial Sequence
  TAU 166 NUCLEIC ACID
EEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQ

SEQ ID NO: 10 - DNA, 498 bp, Artificial Sequence
  FREQUENCY OF H-TAU
gaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccag

SEQ ID NO: 11 - protein, 5 aa, MOUSE
TREPK

SEQ ID NO: 12 - protein, 6 aa, MOUSE
PKSGDR

SEQ ID NO: 13 - protein, 7 aa, MOUSE
PPTREPK

SEQ ID NO: 14 - protein, 12 aa, MOUSE
PPTREPKKVAVV

SEQ ID NO: 15 - protein, 11 aa, MOUSE
PTREPKKVAVV

SEQ ID NO: 16 - protein, 10 aa, MOUSE
GLMVGGVVIA

SEQ ID NO: 17 - protein, 6 aa, MOUSE
EFRHDS

SEQ ID NO: 18 - protein, 42 aa, Homo sapiens [sequence omitted]

SEQ ID NO: 19 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 20 - protein, 16 aa, MOUSE
RSSQNIIHSNGSTYLE

SEQ ID NO: 21 - protein, 7 aa, MOUSE
KVSNRFS

SEQ ID NO: 22 - protein, 9 aa, MOUSE
FQGSHVPWT

SEQ ID NO: 23 - protein, 19 aa, MOUSE
MKLPVRLLVLMFWIPASSS

SEQ ID NO: 24 - protein, 112 aa, MOUSE
DVLMTQTPLSLPVSLGDQASISCRSSQNIIHSNGSTYLEWYLQKPGQSPKLLIYKVSNRFSGVPDRFSGSGSGTDFTLKISRVEAEDLGIYYCFQGSHVPWTFGGGTKLEIK

SEQ ID NO: 25 - DNA, 336 bp, MOUSE
gatgttttgatgacccaaactccactctccctgcctgtcagtcttggagatcaagcctccatctcttgcagatctagtcagaacattatacatagtaatggaagcacctatttagaatggtacctgcagaaaccgggccagtctccaaagctcctgatctacaaagtttccaaccgattttctggggtcccagacaggttcagtggcagtggatcagggacagatttcacactcaagatcagcagagtggaggctgaggatctgggaatttattactgctttcaaggttcacatgttccgtggacgttcggtggaggcaccaagctggaaatcaaa

SEQ ID NO: 26 - protein, 10 aa, MOUSE
GFNIKDEYMN

SEQ ID NO: 27 - protein, 17 aa, MOUSE
WIDPENGDAAYASKFQG

SEQ ID NO: 28 - protein, 11 aa, MOUSE
FYSNYDGYFDV

SEQ ID NO: 29 - protein, 19 aa, MOUSE
MKCSWVIFFLMAVVIGVNS

SEQ ID NO: 30 - protein, 120 aa, MOUSE
EVQLQQSGAELVRPGASVKLSCTASGFNIKDEYMNWVKQRPERGLEWIGWIDPENGDAAYASKFQGKATMTADTSSNTAYLQLSSLTSEDTAVYFCTFFYSNYDGYFDVWGAGTTVTVSS

SEQ ID NO: 31 - protein, 360 aa, MOUSE
GAGGTTCAGCTGCAGCAGTCTGGGGCTGAGCTTGTGAGGCCAGGGGCCTCAGTCAAGTTGTCCTGCACAGCTTCTGGCTTTAACATTAAAGACGAGTATATGAACTGGGTGAAGCAGAGGCCTGAACGGGGCCTGGAGTGGATTGGATGGATTGATCCTGAAAATGGTGATGCTGCATATGCCTCGAAGTTCCAGGGAAAGGCCACTATGACTGCAGACACATCCTCCAACACAGCCTACCTGCAGCTCAGCAGCCTGACATCTGAGGACACTGCCGTCTATTTCTGTACTTTCTTTTACAGTAACTACGACGGGTACTTCGATGTCTGGGGCGCAGGGACCACGGTCACCGTCTCCTCA

SEQ ID NO: 32 - protein, 17 aa, MOUSE
KSSQSLLYSNNQKNYLA

SEQ ID NO: 33 - protein, 7 aa, MOUSE
WASTRES

SEQ ID NO: 34 - protein, 10 aa, MOUSE
QQYYSYPLWT

SEQ ID NO: 35 - protein, 20 aa, MOUSE
MDSQAQVLMLLLLWVSGTCG

SEQ ID NO: 36 - protein, 114 aa, MOUSE
DIVMSQSPSSLAVSIGEKVTMSCKSSQSLLYSNNQKNYLAWYQRKPGQSPKLLIYWASTRESGVPDRFTGSGSGTDFTLTITSVKAEDLAVYYCQQYYSYPLWTFGGGTKLEIK

SEQ ID NO: 37 - DNA, 342 bp, MOUSE
gacattgtgatgtcacagtctccatcctccctagctgtgtcaattggagagaaggttactatgagctgcaagtccagtcagagccttttatatagtaacaatcaaaagaactacttggcctggtaccagcggaaaccagggcagtctcctaaactgctgatttactgggcatccactagggaatctggggtccctgatcgcttcacaggcagtggatctgggacagatttcactctcaccatcaccagtgtgaaggctgaagacctggcagtttattactgtcagcaatattatagttatcctctgtggacgttcggtggaggcaccaagctggaaatcaaa

SEQ ID NO: 38 - protein, 12 aa, MOUSE
GFSLSTSGMGVG

SEQ ID NO: 39 - protein, 16 aa, MOUSE
HIWWDDDKYYNAVLKS

SEQ ID NO: 40 - protein, 11 aa, MOUSE
IGIDGPYAMDY

SEQ ID NO: 41 - protein, 19 aa, MOUSE
MGRLTSSFLLLIVPAYVLS

SEQ ID NO: 42 - protein, 121 aa, MOUSE
QVTLKESGPGILQPSQTLSLTCSFSGFSLSTSGMGVGWIRQPSGKGLEWLAHIWWDDDKYYNAVLKSRLTISKDTSKNQVFLKIASVDTADTATYYCARIGIDGPYAMDYWGQGTSVTVSS

SEQ ID NO: 43 - protein, 363 aa, MOUSE
CAGGTTACTCTGAAAGAGTCTGGCCCTGGGATATTGCAGCCCTCCCAGACCCTCAGTCTGACTTGTTCTTTCTCTGGGTTTTCACTGAGCACTTCTGGTATGGGTGTAGGCTGGATTCGTCAGCCTTCAGGGAAGGGTCTGGAATGGCTGGCACACATTTGGTGGGATGATGATAAGTACTATAACGCAGTCCTGAAGAGCCGGCTCACAATCTCCAAGGATACCTCCAAAAACCAGGTTTTCCTCAAGATCGCCAGTGTGGACACTGCAGATACTGCCACATATTACTGTGCTCGAATAGGGATTGATGGTCCTTATGCTATGGACTACTGGGGTCAAGGAACCTCAGTCACCGTCTCCTCA

(19) United States
(12) Patent Application Publication
(10) Pub. No.: US 2016/0356794 A1
(43) Pub. Date: Dec. 8, 2016

(54) ANTIBODIES THAT BIND TO HUMAN TAU AND ASSAY FOR QUANTIFYING HUMAN TAU USING THE ANTIBODIES

(71) Applicant: MERCK SHARP & DOHME CORP., Rahway, NJ (US)

(72) Inventors: Omar Laterza, New York, NY (US); Michael Tanen, Morganville, NJ (US); Oitak Wong, Livingston, NJ (US)

(73) Assignee: Merck Sharp & Dohme Corp., Rahway, NJ (US)

(21) Appl. No.: 15/117,677

(22) PCT Filed: Feb. 9, 2015
(86) PCT No.: PCT/US15/14976

§ 371 Date: Aug. 9, 2016

Related U.S. Application Data
(60) Provisional application No. 61/937,898, filed on Feb. 10, 2014.

(51) Int. Cl. G01N 33/68; A61K 31/513; A61K 31/549; C07K 16/18

(52) U.S. Cl. CPC G01N 33/6896; C07K 16/18; A61K 31/513; A61K 31/549; G01N 2800/2821; C07K 2317/34; C07K 2317/565; C07K 2317/56; C07K 2317/92; G01N 2333/47; G01N 2333/4709

(57) ABSTRACT

The present invention provides novel antibodies that bind to human Tau and assays for quantifying human Tau using these antibodies.

### FIELD OF THE INVENTION

The present invention relates to antibodies that specifically bind human Tau (h-Tau) and are useful for quantitating h-Tau in biological samples. The invention also relates to assays that employ these antibodies.

### BACKGROUND

Alzheimer's disease (AD), the most common cause of dementia, is a progressive neurodegenerative disorder characterized by increasing loss of memory and cognitive function. Neuropathological features present in AD include amyloid plaques made of Aβ peptides, the most prominent being Aβ₁₋₄₂ peptide, and neurofibrillary tangles (NFTs).

In particular, NFTs consist of paired helical filaments (PHFs) which in turn are composed of the microtubule associated protein h-Tau (h-Tau). Normally h-Tau stabilizes a key cellular network of microtubules that is essential for distributing proteins and nutrients within neurons. In AD patients, however, h-Tau becomes hyperphosphorylated, disrupting its normal functions, increasing its likelihood to aggregate into PHFs and ultimately forming NFTs. It is hypothesized that the formation of NFTs leads to the loss of synapses and neurons, and thus ultimately contributes to the development of dementia.

As the extracellular space of the brain is in direct contact with CSF, biochemical changes in the brain also affect CSF (Blennow et al., The Lancet Neurology, Vol. 2, pp. 605-613, 2003). Studies have shown elevated levels of h-Tau protein in the CSF of AD patients compared with normal subjects (Vandermeeren et al., J. Neurochem., Vol. 61, pp. 1828-1834, 1993; Blennow et al., supra, Hampel et al., Exp. Gerontol, Vol. 45, pp. 30-40, 2010), and thus h-Tau has been used as a biomarker to diagnose AD (Hampel et al. supra). Elevated levels of CSF h-Tau in AD patients have also been shown to correlate with NFT pathology (Tapiola et al., NeuroReport, Vol. 8, pp. 3961-3963, 1997).

Recent studies have also shown that measurement of elevated concentrations of h-Tau in CSF in combination with decreased concentrations of Aβ₁₋₄₂ in CSF can aid in the diagnosis of AD (Tapiola et al., Arch. Neurol., Vol. 66, pp. 382-389, 2009). Further studies have also demonstrated that the ratio of CSF h-Tau/Aβ₁₋₄₂ is useful in identifying individuals with amyloid plaque pathology (Fagan et al., Arch. Neurol., Vol. 68, pp. 1137-1144, 2011). The ratio of CSF h-Tau/Aβ₁₋₄₂ has also been shown to predict future cognitive decline in non-demented older adults and adults having mild AD (Fagan et al., Arch. Neurol., Vol. 64, pp. 343-349, 2007).

In view that the aforementioned CSF biomarkers have been shown to reflect amyloid pathology, neurodegeneration, and are able to prognosticate cognitive decline, they may become important in the identification of asymptomatic or mild symptomatic AD patients, who are most likely to benefit from novel therapeutic interventions.

A requisite for the aforementioned uses of these CSF biomarkers is the accurate quantification of the biomarker present in the CSF of the patient. h-Tau, in particular, is a difficult protein to quantitate for the following reasons. There are six different isoforms of h-Tau varying in size from 352-441 amino acids, all derived from a single gene by alternate mRNA splicing (Himmler et al., Mol. Cell Biol., Vol. 9, pp. 1381-1388, 1989; see FIG. 1). The six h-Tau isoforms differ from one another by the number of (3 or 4) microtubule binding domains and the number of (0, 1, or 2) amino terminal inserts of 29 amino acids each (Goedert et al., Neuron, Vol. 3, pp. 519-526, 1989). The heterogeneity in h-Tau protein is effected by post-translational modifications including phosphorylation, ubiquination, oxidation and others. In addition, h-Tau is present at low concentrations in CSF ranging from about 300 ng/L in healthy individuals to 900 ng/L in AD patients (Blennow and Hampel, Lancet Neurol., Vol. 2, pp. 605-613, 2003).

Immunoassays utilizing monoclonal antibodies have been developed to quantitate h-Tau in CSF (Hampel et al, supra; and Kang et al., Clinical Chem., Vol. 59, pp. 903-916, 2013). Given the molecular heterogeneity and low concentrations of h-Tau in CSF, and the importance of the h-Tau biomarker in the diagnosis of AD in patients at different stages of the disease and its use in to predict future cognitive decline, there remains a continued need to develop highly characterized assays that can accurately quantify all isoforms of h-Tau in CSF.

### SUMMARY OF THE INVENTION

The present invention relates to antibodies, and in particular monoclonal antibodies (mAbs) that specifically bind to epitopes in a region of h-Tau that is conserved in amino acid sequence (amino acids 104-277) in the six known isoforms of h-Tau: h-Tau-441, 412, 410, 383, 381 and 352 (SEQ ID NOs 2 to 7, respectively, as shown in Table 1 below. See also FIG. 1).

TABLE 1
Amino Acid Sequences of: h-Tau Isoforms, Tau 166 Peptide, Aβ₁₋₄₂ Peptide,
Amyloid Beta A4 Protein Isoform A Precursor, Epitopes of h-Tau specifically bound by
mAbs10H8, 19G10 and AT120, and h-Tau Reacting/Non-Reacting with mAbAT120. Nucleic
Acid Sequence encoding Tau 166 Peptide

| Human Tau | | |
|---|---|---|
| Isoform or | | SEQ |
| Peptide | Amino Acid Sequence | ID NO |
| Tau Isoform | MHHHHHHDYDIPTTENLYFQGMAEPRQEWFEVMEDHAGTYGLGDRKDQGGYTMHQDQEGDTDAG | 1 |
| 2-441 | LKESPLQTPTEDGSEEPGSETSDAKSTPTAEDVTAPLVDEGAPGKQAAAQPHTEIPEGTTAEEA | |
| (including HIS | GIGDTPSLEDEAAGHVTQARMVSKSKDGTGSDDKKAKGADGKTKIATPRGAAPPGQKGQANATR | |
| tag in bold | IPAKTPPAPKTPPSSGEPPKSGDRSGYSSPGSPGTPGSRSRTPSLPTPPTREPKKVAVVRTPPK | |
| font and TEV | SPSSAKSRLQTAPVPMPDLKNVKSKIGSTENLKHQPGGGKVQIINKKLDLSNVQSKCGSKDNIK | |
| cleavage site | HVPGGGSVQIVYKPVDLSKVTSKCGSLGNIHHKPGGGQVEVKSEKLDFKDRVQSKIGSLDNITH | |
| in italic font) | VPGGGNKKIETHKLTFRENAKAKTDHGAEIVYKSPVVSGDTSPRHLSNVSSTGSIDMVDSPQLA | |
| | TLADEVSASLAKQGL | |
| Tau Isoform | MAEPRQEFEVMEDHAGTYGLGDRKDQGGYTMHQDQEGDTDAGLKESPLQTPTEDGSEEPGSETS | 2 |
| 2-441 | DAKSTPTAEDVTAPLVDEGAPGKQAAAQPHTEIPEGTTAEEAGIGDTPSLEDEAAGHVTQARMV | |
| Accession No. | SKSKDGTGSDDKKAKGADGKTKIATPRGAAPPGQKGQANATRIPAKTPPAPKTPPSSGEPPKSG | |
| NP 005901.2 | DRSGYSSPGSPGTPGSRSRTPSLPTPPTREPKKVAVVRTPPKSPSSAKSRLQTAPVPMPDLKNV | |
| | KSKIGSTENLKHQPGGGKVQIINKKLDLSNVQSKCGSKDNIKHVPGGGSVQIVYKPVDLSKVTS | |
| | KCGSLGNIHHKPGGGQVEVKSEKLDFKDRVQSKIGSLDNITHVPGGGNKKIETHKLTFRENAKA | |
| | KTDHGAEIVYKSPVVSGDTSPRHLSNVSSTGSIDMVDSPQLATLADEVSASLAKQGL | |
| Tau Isoform | MAEPRQEFEVMEDHAGTYGLGDRKDQGGYTMHQDQEGDTDAGLKESPLQTPTEDGSEEPGSETS | 3 |
| 5-412 | DAKSTPTAEAEEAGIGDTPSLEDEAAGHVTQARMVSKSKDGTGSDDKKAKGADGKTKIATPRGA | |
| (NCBI | APPGQKGQANATRIPAKTPPAPKTPPSSGEPPKSGDRSGYSSPGSPGTPGSRSRTPSLPTPPTR | |
| Accession No. | EPKKVAVVRTPPKSPSSAKSRLQTAPVPMPDLKNVKSKIGSTENLKHQPGGGKVQIINKKLDLS | |
| NP001116539.1) | NVQSKCGSKDNIKHVPGGGSVQIVYKPVDLSKVTSKCGSLGNIHHKPGGGQVEVKSEKLDFKDR | |
| | VQSKIGSLDNITHVPGGGNKKIETHKLTFRENAKAKTDHGAEIVYKSPVVSGDTSPRHLSNVSS | |
| | TGSIDMVDSPQLATLADEVSASLAKQGL | |
| Tau Isoform | MAEPRQEFEVMEDHAGTYGLGDRKDQGGYTMHQDQEGDTDAGLKESPLQTPTEDGSEEPGSETS | 4 |
| 8-410 | DAKSTPTAEDVTAPLVDEGAPGKQAAAQPHTEIPEGTTAEEAGIGDTPSLEDEAAGHVTQARMV | |
| (NCBI | SKSKDGTGSDDKKAKGADGKTKIATPRGAAPPGQKGQANATRIPAKTPPAPKTPPSSGEPPKSG | |
| Accession | DRSGYSSPGSPGTPGSRSRTPSLPTPPTREPKKVAVVRTPPKSPSSAKSRLQTAPVPMPDLKNV | |
| No. NP | KSKIGSTENLKHQPGGGKVQIVYKPVDLSKVTSKCGSLGNIHHKPGGGQVEVKSEKLDFKDRVQ | |
| 001190181.1) | SKIGSLDNITHVPGGGNKKIETHKLTFRENAKAKTDHGAEIVYKSPVVSGDTSPRHLSNVSSTG | |
| | SIDMVDSPQLATLADEVSASLAKQGL | |
| Tau Isoform | MAEPRQEFEVMEDHAGTYGLGDRKDQGGYTMHQDQEGDTDAGLKAEEAGIGDTPSLEDEAAGHV | 5 |
| 3-383 | TQARMVSKSKDGTGSDDKKAKGADGKTKIATPRGAAPPGQKGQANATRIPAKTPPAPKTPPSSG | |
| (NCBI | EPPKSGDRSGYSSPGSPGTPGSRSRTPSLPTPPTREPKKVAVVRTPPKSPSSAKSRLQTAPVPM | |
| Accession | PDLKNVKSKIGSTENLKHQPGGGKVQIINKKLDLSNVQSKCGSKDNIKHVPGGGSVQIVYKPVD | |
| No. NP | LSKVTSKCGSLGNIHHKPGGGQVEVKSEKLDFKDRVQSKIGSLDNITHVPGGGNKKIETHKLTF | |
| 058518.1) | RENAKAKTDHGAEIVYKSPVVSGDTSPRHLSNVSSTGSIDMVDSPQLATLADEVSASLAKQGL | |
| Tau Isoform | MAEPRQEFEVMEDHAGTYGLGDRKDQGGYTMHQDQEGDTDAGLKESPLQTPTEDGSEEPGSETS | 6 |
| 7-381 | DAKSTPTAEAEEAGIGDTPSLEDEAAGHVTQARMVSKSKDGTGSDDKKAKGADGKTKIATPRGA | |
| (NCBI | APPGQKGQANATRIPAKTPPAPKTPPSSGEPPKSGDRSGYSSPGSPGTPGSRSRTPSLPTPPTR | |
| Accession | EPKKVAVVRTPPKSPSSAKSRLQTAPVPMPDLKNVKSKIGSTENLKHQPGGGKVQIVYKPVDLS | |
| No. NP | KVTSKCGSLGNIHHKPGGGQVEVKSEKLDFKDRVQSKIGSLDNITHVPGGGNKKIETHKLTFRE | |
| 001190180.1) | NAKAKTDHGAEIVYKSPVVSGDTSPRHLSNVSSTGSIDMVDSPQLATLADEVSASLAKQGL | |
| Tau Isoform | MAEPRQEFEVMEDHAGTYGLGDRKDQGGYTMHQDQEGDTDAGLKAEEAGIGDTPSLEDEAAGHV | 7 |
| 4-352 | TQARMVSKSKDGTGSDDKKAKGADGKTKIATPRGQQPPGQKGQANATRIPAKTPPAPKTPPSSG | |
| (NCBI | EPPKSGDRSGYSSPGSPGTPGSRSRTPSLPTPPTREPKKVAVVRTPPKSPSSAKSRLQTAPVPM | |
| Accession | PDLKNVKSKIGSTENLKHQPGGGKVQIVYKPVDLSKVTSKCGSLGNIHKHPGGGQVEVKSEKLD | |
| No. NP | FKDRVQSKIGSLDNITHVPGGGNKKIETHKLTFRENAKAKTDHGAEIVYKSPVVSGDTSPRHLS | |
| 058525.1) | NVSSTGSIDMVDSPQLATLADEVSASLAKQGL | |
| Tau 166 peptide | MSYYHHHHHHDYDIPTTENLYFQGEEAGIGDTPSLEDEAAGHVTQARMVSKSKDGTGSDDKKAK | 8 |
| (includeing | GADGKTKIATPRGAAPPGQKGQANATRIPAKTPPAPKTPPSSGEPPKSGDRSGYSSPGSPGTPG | |
| HIS tag in | SRSRTPSLPTPPTREPKKVAVVRTPPKSPSSAKSRLQTAPVPMPDLKNVKSKIGSTENLKHQ | |
| bold font and | | |
| TEV cleavage | | |
| site in italic | | |
| font) | | |
| Tau 166 peptide | EEAGIGDTPSLEDEAAGHVTQARMVSKSKDGTGSDDKKAKGADGKTKIATPRGAAPPGQKGQAN | 9 |
| (AA104-AA269 | ATRIPAKTPPAPKTPPSSGEPPKSGDRSGYSSPGSPGTPGSRSRTPSLPTPPTREPKKVAVVRT | |
| of h-Tau) | PPKSPSSAKSRLQTAPVPMPDLKNVKSKIGSTENLKHQ | |
| Tau 166 | GAAGAAGCAGGCATTGGAGACACCCCCAGCCTGGAAGACGAAGCTGCTGGTCACGTGACCCAAG | 10 |
| nucleic acid | CTCGCATGGTCAGTAAAAGCAAAGACGGGACTGGAAGCGATGACAAAAAAGCCAAGGGGGCTGA | |
| (Sequence | TGGTAAAACGAAGATCGCCACACCGCGGGGAGCAGCCCCTCCAGGCCAGAAGGGCCAGGCCAAC | |
| encoding | GCCACCAGGATTCCAGCAAAAACCCCGCCCGCTCCAAAGACACCACCCAGCTCTGGTGAACCTC | |
| protein in | CAAAATCAGGGGATCGCAGCGGCTACAGCAGCCCCGGCTCCCCAGGCACTCCCGGCAGCCGCTC | |
| bold font) | CCGCACCCCGTCCCTTCCAACCCCACCCACCCGGGAGCCCAAGAAGGTGGCAGTGGTCCGTACT | |
| | CCACCCAAGTCGCCGTCTTCCGCCAAGAGCCGCCTGCAGACAGCCCCCGTGCCCATGCCAGACC | |
| | TGAAGAATGTCAAGTCCAAGATCGGCTCCACTGAGAACCTGAAGCACCAG | |
| Epitope of | TRPEK | 11 |
| 10H8 mAb | | |
| Epitope of | PKSGDR | 12 |
| 19G10 mAb120 | | |
| Epitope of | PPTREPK | 13 |
| mAb AT120 | | |
| described in | | |
| U.S. Pat. No. | | |
| 5,861,257 | | |
| Peptide sequence | PPTREPKKVAVV | 14 |
| reacting with | | |
| mAb AT120 as | | |
| describe in | | |
| U.S. Pat. No. | | |
| 5,861,257 | | |
| Peptide sequence | PTREPKKVAVV | 15 |
| that was | | |
| non-reactive | | |
| with mAb | | |
| AT120 as | | |
| described in | | |
| U.S. Pat. No. | | |
| 5,861,257 | | |
| Epitope of | GLMVGGVVIA | 16 |
| mAb 1-11-3 | | |
| Epitope of | EFRHDS | 17 |
| mAb 6E10 | | |
| Aβ₁₋₄₂ | DAEFRHDSGYEVHHQKLVFFAEDVGSNKGAIIGLMVGGVVIA | 18 |
| Peptide | | |
| Amyloid beta | MLPGLALLLLAAWTARALEVPTDGNAGLLAEPQIAMFCGRLNMHMNVQNGKWDSDPSGTKTCID | 19 |
| A4 protein | TKEGILQYCQEVYPELQITNVVEANQPVTIQNWCKRGRKQCKTHPHFVIPYRCLVGEFVSDALL | |
| isoform a | VPDKCKFLHQERMDVCETHLHWHTVAKETCSEKSTNLHDYGMLLPCGIDKFRGVEFVCCPLAEE | |
| presursor | SDNVDSADAEEDDSDVWWGGADTDYADGSEDKVVEVAEEEEVAEVEEEEADDDEDDEDGDEVEE | |
| [Homo sapiens] | EAEEPYEEATERTTSIATTTTTTTESVEEVVREVCSEQAETGPCRAMISRWYFDVTEGKCAPFF | |
| (NCBI | YGGCGGNRNNFDTEEYCMAVCGSAMSQSLLKTTQEPLARDPVKLPTTAASTPDAVDKYLETPGD | |
| Accession No. | ENEHAHFQKAKERLEAKHRERMSQVMREWEEAERQAKNLPKADKKAVIQHFQEKVESLEQEAAN | |
| NP_000465.1) | ERQQLVETHMARVEAMLNDRRRLALENYITALQAVPPRPRHVFNMLKKYVRAEQKDRQHTLKHF | |
| | EHVRMVDPKKAAQIRSQVMTHLRVIYERMNQSLSLLYNVPAVAEEIQDEVDELLQKEQNYSDDV | |
| | LANMISEPRISYGNDALMPSLTETKTTVELLPVNGEFSLDDLQPWHSFGADSVPANTENEVEPV | |
| | DARPAADRGLTTRPGSGLTNIKTEEISEVKMDAEFRHDSGYEVHHQKLVFFAEDVGSNKGAIIG | |
| | LMVGGVVIATVIVITLVMLKKKQYTSIHHGVVEVDAAVTPEERHLSKMQQNGYENPTYKFFEQM | |
| | QN | |

The inventors were concerned with developing a pair of antibodies that when used together in a h-Tau assay would possess both the requisite sensitivity to quantitate all the six isoforms of h-Tau in CSF (analytic performance) and the ability to differentiate AD patients from healthy controls (diagnostic performance), in particular for the purpose of selecting patients for treatment with an AD therapeutic agent. To generate a pair of antibodies that would possess the aforementioned characteristics, three immunogens were employed: h-Tau 441, h-Tau 352, and Tau 166 peptide, which is a synthetic peptide spanning amino acids 104 to 269 (SEQ ID NO: 9) of the conserved region of h-Tau. The inventors found that in utilizing Tau 166 peptide as an immunogen, more parental clones were generated having antibodies that specifically bound to the conserved region of h-Tau compared to the amount of parental clones generated utilizing the other two immunogens. In particular, upon screening the clones for antibodies specific for the conserved region of h-Tau, two novel mAbs 10H8 and 19G10 were identified that were specific for the conserved region and which were generated using the Tau 166 peptide as immunogen. The mAb 10H8 specifically binds to a five amino acid epitope, TREPK (SEQ ID NO: 11) corresponding to amino acids 220 to 224 of h-Tau. The mAb 19G10 specifically binds to a six amino acid epitope, PKSGDR (SEQ ID NO: 12) corresponding to amino acids 189 to 194 of h-Tau (See also Tables 2 and 3 for amino acid sequences of epitopes of mAbs 10H8 and 19G10, respectively). In particular, the inventors found that when pairing mAb 10H8 as the capture antibody and mAb 19G10 as the detection antibody in a h-Tau assay, the mAbs 10H8 and 19G10 paired in this manner provided both the highest clinical sensitivity and specificity, among 8 different antibody pairs (Example 1), in terms of quantitating all isoforms of h-Tau in CSF, and the best ability to differentiate AD patients from normal, as compared to other paired mAbs generated against h-Tau. While it is known to utilize the isoforms h-Tau 441 and h-Tau 352 as immunogens for production of antibodies, it is believed to be the first time that Tau 166 peptide has been used as an immunogen to selectively generate antibodies that recognize all isoforms of h-Tau and thus allow their quantitation in CSF.

In one aspect, the present invention provides an isolated antibody or antigen binding fragment thereof that specifically binds an epitope of h-Tau consisting of amino acids 220 to 224 (SEQ ID NO: 11).

In one embodiment, the isolated antibody or antigen binding fragment binding to the epitope consisting of amino acids 220 to 224 of h-Tau comprises three light chain CDRs of SEQ ID NO: 20 (CDRL1), SEQ ID NO: 21 (CDRL2) and SEQ ID NO: 22 (CDRL3) and three heavy chain CDRs of SEQ ID NO: 26 (CDRH1), SEQ ID NO: 27 (CDRH2) and SEQ ID NO: 28 (CDRH3) or a variant of the antibody. In one embodiment, the variant of the antibody comprises 1, 2, 3, 4, 5, and 6 amino acid substitutions in one or more of the above recited CDRs, but retains the ability to bind an epitope of h-Tau consisting of amino acids 220 to 224 (SEQ ID NO: 11).

In another embodiment, the isolated antibody or antigen binding fragment binding to the epitope consisting of amino acids 220 to 224 of h-Tau comprises a light chain variable region of SEQ ID NO: 24 and a heavy chain variable region of SEQ ID NO: 30 or a variant of the antibody. In one embodiment, the variant of the antibody comprises 1-20 amino acid substitutions in one or both of these sequences, but retains the ability to bind an epitope of h-Tau consisting of amino acids 220 to 224 (SEQ ID NO: 11).

In another aspect, the present invention provides an isolated antibody or antigen binding fragment thereof that specifically binds an epitope of h-Tau consisting of amino acids 189 to 194 (SEQ ID NO: 12).

In another embodiment, the isolated antibody or antigen binding fragment thereof binding to the epitope consisting of amino acids 189 to 194 of h-Tau comprises three light chain CDRs of SEQ ID NO: 32 (CDRL1), SEQ ID NO: 33 (CDRL2) and SEQ ID NO: 34 (CDRL3) and three heavy chain CDRs of SEQ ID NO: 38 (CDRH1), SEQ ID NO: 39 (CDRH2) and SEQ ID NO: 40 (CDRH3) or a variant of the antibody. In one embodiment, the variant of the antibody comprises 1, 2, 3, 4, 5, and 6 amino acid substitutions in one or more of the above recited CDRs, but retains the ability to bind an epitope of h-Tau consisting of amino acids 189 to 194 (SEQ ID NO: 12).

In another embodiment, the isolated antibody or antigen binding fragment thereof binding to the epitope consisting of amino acids 189 to 194 of h-Tau comprises a light chain variable domain of SEQ ID NO: 36 and a heavy chain variable domain of SEQ ID NO: 42 or a variant of the antibody. In one embodiment, the variant of the antibody comprises 1-20 amino acid substitutions in one or both of these sequences, but retains the ability to bind an epitope of h-Tau consisting of amino acids 189 to 194 (SEQ ID NO: 12).

In other aspects, the present invention provides nucleic acids encoding the variable light and heavy chains of these antibodies, expression vectors comprising these nucleic acids, host cells comprising the expression vectors, and methods for producing the antibody or antigen binding fragment thereof.

In another aspect, the present invention provides an isolated Tau 166 peptide (SEQ ID NO: 9) for use, in particular, as an immunogen for producing the antibodies of the present invention. In an embodiment, the present invention also provides host cells transfected with a nucleic acid encoding the Tau 166 peptide.

In another aspect, the present invention provides methods for quantitating h-Tau utilizing one or both of the aforementioned antibodies and kits comprising these antibodies for use in diagnosing AD and selecting AD patients for treatment with an AD therapeutic agent, e.g., a BACE-1 inhibitor.

In an embodiment, the present invention provides a method for diagnosing Alzheimer's disease in a patient suspected of having this disease, the method comprising:
(a) quantifying the amount of human Tau in a cerebrospinal fluid sample of the patient by:
(1) capturing human Tau from the sample by contacting the sample with an antibody or antigen binding fragment thereof, specifically binding to the epitope consisting of amino acids 220-224 of h-Tau, selected from the group consisting of:
   (i) an antibody or antigen binding fragment thereof comprising three light chain CDRs of SEQ ID NO: 20 (CDRL1), SEQ ID NO: 21 (CDRL2) and SEQ ID NO: 22 (CDRL3) and three heavy chain CDRs of SEQ ID NO: 26 (CDRH1), SEQ ID NO: 27 (CDRH2) and SEQ ID NO: 28 (CDRH3) or a variant of the antibody, and
   (ii) an isolated antibody or antigen binding fragment thereof comprising a light chain variable region of SEQ ID NO: 24 and a heavy chain variable region of SEQ ID NO: 30 or a variant of the antibody, under conditions allowing formation of a capture antibody/Tau complex, wherein the antibody or antigen binding fragment is immobilized onto a solid support; and
(2) detecting the captured Tau by contacting the capture antibody/Tau complex with a detectably labeled antibody or antibody fragment, specifically binding to the epitope consisting of amino acids 189 to 194, selected from the group consisting of:
   (i) an antibody or antigen binding fragment thereof comprising three light chain CDRs of SEQ ID NO: 32 (CDRL1), SEQ ID NO: 33 (CDRL2) and SEQ ID NO: 34 (CDRL3) and three heavy chain CDRs of SEQ ID NO: 38 (CDRH1), SEQ ID NO: 39 (CDRH2) and SEQ ID NO: 40 (CDRH3) or a variant of the antibody, and
   (ii) an antibody or antigen binding fragment thereof comprising a light chain variable domain of SEQ ID NO: 36 and a heavy chain variable domain of SEQ ID NO: 42 or a variant of the antibody, under conditions allowing formation of a capture antibody/Tau/detectable labeled antibody complex; and
(b) determining the concentration of human Tau in step (a), wherein a value greater than 184 pg/mL indicates a diagnosis of AD in the patient.

In another embodiment of the aforementioned method of diagnosing Alzheimer's disease in a patient suspected of having this disease, the method further comprises
(c) quantifying the amount of Aβ₁₋₄₂ in the cerebrospinal fluid sample of the patient; and
(d) determining the ratio of human Tau/Aβ₁₋₄₂ in the sample of the patient, wherein a ratio value greater than 0.215 indicates a diagnosis of AD in the patient. In yet another aspect, a method of treating Alzheimer's disease in a patient in need thereof is provided. The method comprises:
   (a) selecting a patient in need of treatment using the aforementioned diagnostic methods; and
   (b) administering to the patient a therapeutically effective amount of an AD therapeutic agent.
      In an embodiment of the aforementioned method of treating Alzheimer's disease, the AD therapeutic agent is a BACE-1 inhibitor having the structure

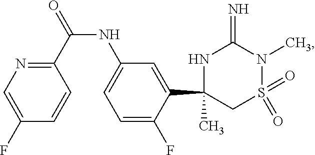 [SMILES: CN1C(=N)N[C@](C)(c2cc(NC(=O)c3ccc(F)cn3)ccc2F)CS1(=O)=O]

a tautomer thereof, or a pharmaceutically acceptable salt of the compound or the tautomer.

### BRIEF DESCRIPTION OF THE DRAWINGS

FIG. 1 shows the amino acid sequences (SEQ ID NOS 2 to 7) of the six known h-Tau isoforms (h-Tau 441, h-Tau 412, h-Tau 410, h-Tau-381, h-Tau 383, and h-Tau 352, respectively. The epitopes for mAbs 10H8 (epitope TREPK, SEQ ID NO: 11, corresponding to amino acids 220 to 224 of h-Tau) and 19G10 (epitope PKSGDR, SEQ ID NO: 12, corresponding to amino acids 189 to 194 of h-Tau) are in bolded brackets.

FIG. 2 shows the estimated ROC curves (100*Sensitivity vs. 100*(1-Specificity)) for CSF Aβ42, tau, and tau/Aβ42. Reference lines are drawn at 80% sensitivity and 60% specificity.

FIG. 3 displays estimates of sensitivity, specificity, and total agreement with PET (Flutemetamol visual read) vs. prospective thresholds for CSF h-Tau/Aβ42 using log scaling. Sensitivity (solid line) and Specificity (solid line) are displayed along with 95% lower confidence limits (dashed lines). The estimate of Total Agreement (solid line) is based on nonparametric density estimation. Vertical lines (solid lines) show the CSF window (0.169, 0.360) that achieves the acceptable sensitivity and specificity performance. The value that maximizes total agreement within this window (0.215) is also shown with a vertical line and identified on the top axis.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“Aβ₁₋₄₂ peptide or Aβ₁₋₄₂” refers to a 42 amino acid peptide corresponding to amino acids 672 to 713 (SEQ ID NO: 18) which is produced by proteolytic cleavage of the amyloid beta A4 protein isoform a precursor protein (SEQ ID NO: 19) by the β- and γ-secretases.

“Administration” or “administering” an AD therapeutic agent means providing an AD therapeutic agent to the patient in need of treatment.

“Alzheimer's disease or AD” as used herein refers to the spectrum of dementias or cognitive impairment resulting from neuronal degradation associated with the formation or deposition of Aβ plaques or NFTs in the brain, from the spectrum of Alzheimer's disease including but not limited to preclinical Alzheimer's disease, mild cognitive impairment due to Alzheimer's disease, early onset Alzheimer's disease, familial Alzheimer's disease, through the advance cognitive impairment of dementia due to Alzheimer's disease (Jack et al., Alzheimer's Dement., May 7 (3), pp. 257-262, 2011) and diseases associated with the presence of the ApoE4 allele.

“AD therapeutic agent” as used herein refers to a treatment or intervention that addresses one or more underlying pathophysiologies of AD or a symptom thereof.

Examples of AD therapeutic agents include, but are not limited to, the BACE-1 inhibitors described herein, BACE-1 inhibitors CTS-21166 (CoMentis Inc.), AZD3293 (AstraZeneca), E-2609 (Eisai), TAK-070 (Takeda), and HPP-854 (Transtech), gamma secretase inhibitors (e.g., as described in WO2007/084595 and WO2009/008980), gamma secretase modulators (as described e.g., in WO2008/153793 and WO2010/056849), solanezumab (Eli Lilly), liraglutide (Lancaster University), bexarotene (brand name Targretin®), ACC-001 (vaccine), muscarinic antagonists (e.g., m₁ agonists (such as acetylcholine, oxotremorine, carbachol, or McNa343), or m₂ antagonists (such as atropine, dicycloverine, tolterodine, oxybutynin, ipratropium, methoctramine, tripitamine, or gallamine); cholinesterase inhibitors (e.g., acetyl- and/or butyrylchlolinesterase inhibitors such as donepezil (Aricept®), galantamine (Razadyne®), and rivastigimine (Exelon®); N-methyl-D-aspartate receptor antagonists (e.g., Namenda® (memantine HCl, available from Forrest Pharmaceuticals, Inc.); combinations of cholinesterase inhibitors and N-methyl-D-aspartate receptor antagonists; non-steroidal anti-inflammatory agents; anti-inflammatory agents that can reduce neuroinflammation; anti-amyloid antibodies (such as bapineuzemab, Wyeth/Elan); vitamin E; nicotinic acetylcholine receptor agonists; CB 1 receptor inverse agonists or CB 1 receptor antagonists; antibiotics; growth hormone secretagogues; histamine H3 antagonists; AMPA agonists; PDE4 inhibitors; GABA_{A} inverse agonists; inhibitors of amyloid aggregation; glycogen synthase kinase beta inhibitors; promoters of alpha secretase activity; PDE-10 inhibitors; Tau kinase inhibitors (e.g., GSK3beta inhibitors, cdk5 inhibitors, or ERK inhibitors); Tau aggregation inhibitors (e.g., Rember®); RAGE inhibitors (e.g., TTP 488 (PF-4494700)); anti-Abeta vaccine; APP ligands; agents that upregulate insulin, cholesterol lowering agents such as HMG-CoA reductase inhibitors (for example, statins such as Atorvastatin, Fluvastatin, Lovastatin, Mevastatin, Pitavastatin, Pravastatin, Rosuvastatin, Simvastatin) and/or cholesterol absorption inhibitors (such as Ezetimibe), or combinations of HMG-CoA reductase inhibitors and cholesterol absorption inhibitors (such as, for example, Vytorin®); fibrates (such as, for example, clofibrate, Clofibride, Etofibrate, and Aluminium Clofibrate); combinations of fibrates and cholesterol lowering agents and/or cholesterol absorption inhibitors; nicotinic receptor agonists; niacin; combinations of niacin and cholesterol absorption inhibitors and/or cholesterol lowering agents (e.g., Simcor® (niacin/simvastatin, available from Abbott Laboratories, Inc.); LXR agonists; LRP mimics; H3 receptor antagonists; histone deacetylase inhibitors; hsp90 inhibitors; 5-HT4 agonists (e.g., PRX-03140 (Epix Pharmaceuticals)); 5-HT6 receptor antagonists; mGluR1 receptor modulators or antagonists; mGluR5 receptor modulators or antagonists; mGluR2/3 antagonists; Prostaglandin EP2 receptor antagonists; PAI-1 inhibitors; agents that can induce Abeta efflux such as gelsolin; Metal-protein attenuating compound (e.g, PBT2); and GPR3 modulators; and antihistamines such as Dimebolin (e.g., Dimebon®, Pfizer).

“Antibody” as used herein may refer to any form of antibody that exhibits the desired biological activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to those variable domain residues other than the hypervariable region residues defined herein as CDR residues.

As used herein, antibody 10H8 is the antibody produced by hybridoma subclone MEB clone 10H8.25.6.10H8 (murine IgG1 isotype) comprising the light chain and heavy chain variable regions (SEQ ID NOs: 24 and 30, respectively) set forth in Table 2 below.

TABLE 2
Characteristics of Monoclonal Antibody 10H8

| Antibody Feature | Amino Acid Sequence or Nucleic Acid Sequence | SEQ ID NO |
|---|---|---|
| Light Chain | | |
| CDRL1 | RSSQNIIHSNGSTYLE | 20 |
| CDRL2 | KVSNRFS | 21 |
| CDRL3 | FQGSHVPWT | 22 |
| Leader | MKLPVRLLVLMFWIPASSS | 23 |
| Sequence | | |
| Variable Region | DVLMTQTPLSLPVSLGDQASISCRSSQNIIHSNGSTYLEQYLQKPGQSPKLLIYKV | 24 |
| (CDRs in bold | SNRFSGVPDRFSGSGSGTDFTLKISRVEAEDLGIYYCFQGSHVPWTFGGGTKLEIK | |
| font and FRs | | |
| in italic font) | | |
| DNA Sequence | GATGTTTTGATGACCCAAACTCCACTCTCCCTGCCTGTCAGTCTTGGAGATCAAGC | 25 |
| Encoding the | CTCCATCTCTTGCAGATCTAGTCAGAACATTATACATAGTAATGGAAGCACCTATT | |
| Variable Region | TAGAATGGTACCTGCAGAAACCGGGCCAGTCTCCAAAGCTCCTGATCTACAAAGTT | |
| (CDRs in bold | TCCAACCGATTTTCTGGGGTCCCAGACAGGTTCAGTGGCAGTGGATCAGGGACAGA | |
| font and FRs in | TTTCACACTCAAGATCAGCAGAGTGGAGGCTGAGGATCTGGGAATTTATTACTGCT | |
| italic font) | TTCAAGGTTCACATGTTCCGTGGACGTTCGGTGGAGGCACCAAGCTGGAAATCAAA | |
| Heavy Chain | | |
| CDRH1 | GFNIKDEYMN | 26 |
| CDRH2 | WIDPENGDAAYASKFQG | 27 |
| CDRH3 | FYSNYDGYFDV | 28 |
| Leader Sequence | MKCSWVIFFLMAVVIGVNS | 29 |
| Variable Region | EVQLQQSGAELVRPGASVKLSCTASGFNIKDEYMNWVKQRPERGLEWIGWIDPENG | 30 |
| (CDRs in bold | DAAYASKFQGKATMTADTSSNTAYLQLSSLTSEDTAVYFCTFFYSNYDGYFDVWGA | |
| font and FRs in | GTTVTVSS | |
| italic font) | | |
| DNA Sequence | GAGGTTCAGCTGCAGCAGTCTGGGGCTGAGCTTGTGAGGCCAGGGGCCTCAGTCAA | 31 |
| Encoding the | GTTGTCCTGCACAGCTTCTGGCTTTAACATTAAAGACGAGTATATGAACTGGGTGA | |
| Variable Region | AGCAGAGGCCTGAACGGGGCCTGGAGTGGATTGGATGGATTGATCCTGAAAATGGT | |
| (CDRs in bold | GATGCTGCATATGCCTCGAAGTTCCAGGGAAAGGCCACTATGACTGCAGACACATC | |
| font and FRs in | CTCCAACACAGCCTACCTGCAGCTCAGCAGCCTGACATCTGAGGACACTGCCGTCT | |
| italic font) | ATTTCTGTACTTTCTTTTACAGTAACTACGACGGGTACTTCGATGTCTGGGGCGCA | |
| | GGGACCACGGTCACCGTCTCCTCA | |

As used herein, antibody 19G10 is the antibody produced by hybridoma subclone MEB.19G10.10.5 (murine isotype IgG2b) comprising the light chain and heavy chain variable regions (SEQ ID NOs: 36 and 42, respectively) set forth in Table 3 below.

TABLE 3
Characteristics of Monoclonal Antibody 19G10

| Antibody Feature | Amino Acid Sequence or Nucleic Acid Sequence | SEQ ID NO |
|---|---|---|
| Light Chain | | |
| CDRL1 | KSSQSLLYSNNQKNYLA | 32 |
| CDRL2 | WASTRES | 33 |
| CDRL3 | QQYYSYPLWT | 34 |
| Leader Sequence | MDSQAQVLMLLLLWVSGTCG | 35 |
| Variable Region | DIVMSQSPSSLAVSIGEKVTMSCKSSQSLLYSNNQKNYLAWYQR | 36 |
| (CDRs in bold | KPGQSPKLLIYWASTRESGVPDRFTGSGSGTDFTLTITSVKAED | |
| font and Frs in | LAVYYCQQYYSYPLWTFGGGTKLEIK | |
| italic font) | | |
| DNA Sequence | GACATTGTGATGTCACAGTCTCCATCCTCCCTAGCTGTGTCAAT | 37 |
| Encoding the | TGGAGAGAAGGTTACTATGAGCTGCAAGTCCAGTCAGAGCCTTT | |
| Variable Region | TATATAGTAACAATCAAAAGAACTACTTGGCCTGGTACCAGCGG | |
| (CDRs in bold | AAACCAGGGCAGTCTCCTAAACTGCTGATTTACTGGGCATCCAC | |
| font and FRs in | TAGGGAATCTGGGGTCCCTGATCGCTTCACAGGCAGTGGATCTG | |
| italic font) | GGACAGATTTCACTCTCACCATCACCAGTGTGAAGGCTGAAGAC | |
| | CTGGCAGTTTATTACTGTCAGCAATATTATAGTTATCCTCTGTG | |
| | GACGTTCGGTGGAGGCACCAAGCTGGAAATCAAA | |
| Heavy Chain | | |
| CDRH1 | GFSLSTSGMGVG | 38 |
| CDRH2 | HIWWDDDKYYNAVLKS | 39 |
| CDRH3 | IGIDGPYAMDY | 40 |
| Leader Sequence | MGRLTSSFLLLIVPAYVLS | 41 |
| Variable Region | QVTLKESGPGILQPSQTLSLTCFSGFSLSTSGMGVGWIRQPSGK | 42 |
| (CDRs in bold | GLEWLAHIWWDDDKYYNAVLKSRLTISKDTSKNQVFLKIASVDT | |
| font and FRs in | ADTATYYCARIGIDGPYAMDYWGQGTSVTVSS | |
| italic font) | CAGGTTACTCTGAAAGAGTCTGGCCCTGGGATATTGCAGCCCTC | |
| DNA Sequence | CCAGACCCTCAGTCTGACTTGTTCTTTCTCTGGGTTTTCACTGA | 43 |
| Encoding the | GCACTTCTGGTATGGGTGTAGGCTGGATTCGTCAGCCTTCAGGG | |
| Variable Region | AAGGGTCTGGAATGGCTGGCACACATTTGGTGGGATGATGATAA | |
| (CDRs in bold | GTACTATAACGCAGTCCTGAAGAGCCGGCTCACAATCTCCAAGG | |
| font and FRs in | ATACCTCCAAAAACCAGGTTTTCCTCAAGATCGCCAGTGTGGAC | |
| italic font) | ACTGCAGATACTGCCACATATTACTGTGCTCGAATAGGGATTGA | |
| | TGGTCCTTATGCTATGGACTACTGGGGTCAAGGAACCTCAGTCA | |
| | CCGTCTCCTCA | |

As used herein an antibody is said to “specifically bind to an epitope on h-Tau” if it binds to that epitope on the known six isoforms of h-Tau, but does not bind to other epitopes on h-Tau.

As used herein an antibody is said to “specifically bind to an epitope on the N-terminal or C-terminal of Aβ₁₋₄₂” if it binds to that epitope but does not bind to other epitopes on AN-42.

As used herein “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; Nanobodies® and multispecific antibodies formed from antibody fragments.

In an embodiment, the antibody or antigen binding fragment comprises a heavy chain constant region, e.g. a human constant region, such as γ1, γ2, γ3, or γ4 human heavy chain constant region or a variant thereof. In another embodiment, the antibody or antigen binding fragment comprises a light chain constant region, e.g. a human light chain constant region, such as lambda or kappa human light chain region or variant thereof. By way of example, and not limitation the human heavy chain constant region can be yl and the human light chain constant region can be kappa.

“Biological sample” as used herein refers to any type of fluid or tissue sample. Typical examples that may be used in the assays herein are whole blood, plasma, serum, urine, cerebral spinal fluid (CSF) and extracts of brain tissue.

“Capture antibody” as used herein refers to an antibody that is used in the disclosed assays to retrieve from a biological sample all the isoforms making up h-Tau. In one aspect, the capture antibody as used herein specifically binds to the epitope on h-Tau consisting of amino acids TREPK (amino acids 220 to 224, SEQ ID NO: 11). In an embodiment, the capture antibody binding to the aforementioned epitope on h-Tau is the mAb 10H8. In another aspect, the capture antibody as used herein specifically binds to an epitope of the N-terminal and/or C-terminal of Aβ₁₋₄₂. In one embodiment, the capture antibody specifically binds to an epitope on the C-terminal of Aβ₁₋₄₂ comprising amino acids GLMVGGVVIA (SEQ ID NO: 16, corresponding to amino acids 33 to 42 of SEQ ID NO: 18). In another embodiment, the capture antibody binding to the aforementioned epitope on Aβ₁₋₄₂ is rabbit mAb 1-11-3.

The phrase “control sequences” as used herein refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

“Detectably labeled antibody” refers to an antibody that is labeled with a reagent capable of detecting the antibody. The reagent may include, but is not limited to, a radioactive isotope, an enzyme, a biotin, dye, fluorescent label and chemiluminescent label as set forth below. The “detectably labeled antibody” is used to detect the amount of h-Tau or Aβ₁₋₄₂ which has been retained by the capture antibody. In one aspect, the detectably labeled antibody as used herein specifically binds to an epitope on h-Tau consisting of amino acids 189 to 194 (PKSGDR, SEQ ID NO: 12). In an embodiment, the detectably labeled antibody specifically binding to the aforementioned epitope on h-Tau is the mAb 19G10. In another aspect, the detectably labeled antibody as used herein specifically binds to an epitope on the N-terminal and/or C-terminal of AB₁₋₄₂. In an embodiment, the detectably labeled antibody specifically binds to an epitope on the N-terminal of Aβ₁₋₄₂ comprising amino acids EFRHDS (amino acids 3 to 8, SEQ ID NO:17). In another embodiment, the detectably labeled antibody binding to the aforementioned epitope of Aβ₁₋₄₂ is mAb 6E10.

“Diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

“Epitope” refers to the segment of amino acids on h-Tau capable of being recognized by, and bound by, an anti-h-Tau antibody of the present invention or other anti-h-Tau antibody, or a segment of amino acids on Aβ₁₋₄₂ capable of being recognized by, and bound by, an antibody.

A “Fab fragment” is comprised of one light chain and the C_{H}1 and variable regions of one heavy chain. The heavy chain of a Fab molecule cannot form a disulfide bond with another heavy chain molecule. A “Fab fragment” can be the product of papain cleavage of an antibody.

An “Fc” region contains two heavy chain fragments comprising the C_{H}1 and C_{H}2 domains of an antibody. The two heavy chain fragments are held together by two or more disulfide bonds and by hydrophobic interactions of the C_{H}3 domains.

A “Fab′ fragment” contains one light chain and a portion or fragment of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

A “F(ab′)₂ fragment” contains two light chains and two heavy chains containing a portion of the constant region between the C_{H}1 and C_{H}² domains, such that an interchain disulfide bond is formed between the two heavy chains. A F(ab′)₂ fragment thus is composed of two Fab′ fragments that are held together by a disulfide bond between the two heavy chains. An “F(ab′)₂ fragment” can be the product of pepsin cleavage of an antibody.

The “Fv region” comprises the variable regions from both the heavy and light chains, but lacks the constant regions.

“h-Tau” as used herein refers to h-Tau which includes the six known isoforms of h-Tau. Quantification of h-Tau refers to the amount of h-Tau obtained from the six known isoforms of h-Tau.

“Isolated antibody” refers to the purification status and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Kd” as used herein refers to the “dissociation constant” of a particular antibody-antigen interaction as is known in the art.

The term “monoclonal antibody or mAb”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Polyclonal antibody” refers to an antibody which was produced among or in the presence of one or more other, non-identical antibodies. In general, polyclonal antibodies are produced from collections of different B-lymphocytes, e.g. the B-lymphocyte of an animal treated with an immunogen of interest, which produces a population of different antibodies that are all directed to the immunogen. Usually, polyclonal antibodies are obtained directly from an immunized animal, e.g. spleen, serum or ascites fluid.

The term “salt(s)”, as employed herein, denotes acidic salts formed with inorganic and/or organic acids, as well as basic salts formed with inorganic and/or organic bases. In addition, when a compound of the invention contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the BACE-1 inhibitors described herein may be formed, for example, by reacting the BACE-1 inhibitor with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts and all acid and base salts are considered equivalent to the free forms of the corresponding BACE-1 inhibitor described herein.

The term “single-chain Fv” or “scFv” antibody refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

The term “treatment” or “treating” means any administration of an AD therapeutic agent to obtain a desired pharmacologic and/or physiologic effect. The effect may be prophylactic in terms of completely or partially preventing a disease or symptom thereof, and/or may be therapeutic in terms of a partial or complete cure for a disease and/or adverse effect attributable to the disease. Treatment includes (1) inhibiting the disease in a patient, e.g., a human, that is experiencing or displaying the pathology or symptomatology of the disease (i.e., arresting further development of the pathology and/or symptomatology), or (2) ameliorating the disease in a patient that is experiencing or displaying the pathology or symptomatology of the disease (i.e., reversing the pathology and/or symptomatology).

The amount of an AD therapeutic agent that is effective to alleviate any particular disease symptom (also referred to as the “therapeutically effective amount”) may vary according to factors such as the disease state, age, and weight of the patient, and the ability of the drug to elicit a desired response in the subject or patient. Whether a disease symptom has been alleviated can be assessed by any clinical measurement typically used by physicians or other skilled healthcare providers to assess the severity or progression status of that symptom.

### Physical and Functional Properties of the Exemplary Anti-h-Tau Antibodies and Antigen-Binding Fragments

The present invention provides isolated anti-h-Tau antibodies and antigen binding fragments thereof and methods of quantifying h-Tau in a biological sample such as CSF using these antibodies and antigen binding fragments thereof. Examples of the anti-h-Tau antibodies of the present invention include but are not limited to: mAbs 101-18 (see Table 2, light chain and heavy chain variable regions of SEQ ID NOs: 24 and 30, respectively) of murine isotype IgG1, and 19G10 (see Table 3, light chain and heavy chain variable regions of SEQ ID NOs: 36 and 42, respectively) of murine isotype IgG2b.

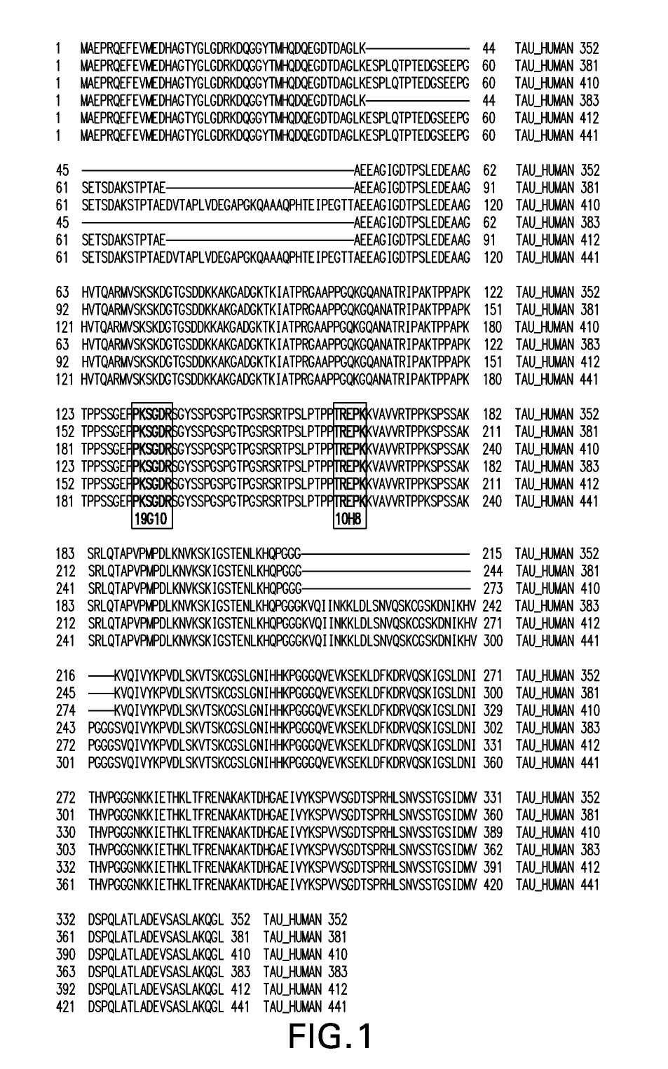

The 10H8 and 19G10 antibodies specifically bind non-identical epitopes located in a conserved region shared by all six isoforms of h-Tau, which spans amino acids 104 to 277 of h-Tau (See FIG. 1).

In one aspect, an isolated antibody or antigen binding fragment thereof is provided which specifically binds an epitope on h-Tau consisting of amino acids 220 to 224 (TREPK) as set forth in SEQ ID NO: 11. U.S. Pat. No. 5,861,257 describes mAb AT120 which specifically binds to an epitope on h-Tau comprising amino acids PPTREPK (SEQ ID NO: 13) corresponding to amino acids Pro 218 to Lys 224 of h-Tau. The antibody of the present invention which specifically binds to epitope TREPK (SEQ ID NO: 11), as exemplified by mAb 10H8 is thought to be a different antibody from mAb AT120 described in U.S. Pat. No. 5,861,257 in view of the difference in their respective epitopes. In this regard, epitope mapping (see paragraph bridging columns 19-20 of U.S. Pat. No. 5,861,257) of mAb AT120 indicated that while mAb AT120 reacted with the peptide sequence, PPTREPKKVAVV (SEQ ID NO: 14), mAb AT120 did not react with the peptide sequence, PTREPKKVAVV (SEQ ID NO: 15). These and additional peptide mapping results of mAb AT120 indicated that the epitope specifically bound by mAb AT120 was PPTREPK (SEQ ID NO: 13), and not the epitope TREPK (SEQ ID NO: 11) specifically bound by the antibody of the present invention as exemplified by mAb 10H8 (see Example 2 which shows epitope mapping results for mAb 10H8).

In another embodiment, the isolated antibody or antigen binding fragment specifically binding to the epitope of SEQ ID NO: 11 (TREPK) comprises three light chain CDRs of SEQ ID NO: 20 (CDRL1), SEQ ID NO: 21 (CDRL2) and SEQ ID NO: 22 (CDRL3) and three heavy chain CDRs of SEQ ID NO: 26 (CDRH1), SEQ ID NO: 27 (CDRH2) and SEQ ID NO: 28 (CDRH3) or a variant of the antibody. In one embodiment, the variant of the antibody comprises, 1, 2, 3, 4, 5, and 6 amino acid substitutions in one or more of the above recited CDRs, but retains the ability to bind an epitope of h-Tau consisting of amino acids 220 to 224 (SEQ ID NO: 11).

In another embodiment, the isolated antibody or antigen binding fragment thereof binding to the epitope of SEQ ID NO: 11 (TREPK) comprises a light chain variable region of SEQ ID NO: 24 and a heavy chain variable region of SEQ ID NO: 30 or a variant of the antibody. In another embodiment, the variant of the antibody comprises 1-20 amino acid substitutions in one or both sequences, but retains the ability to bind an epitope of h-Tau consisting of amino acids 220 to 224 (SEQ ID NO: 11).

In another embodiment, the isolated antibody or antigen binding fragment thereof binding to the epitope of SEQ ID NO: 11 (TREPK) is a mAb or antigen binding fragment thereof. In a particularly useful embodiment, the mAb is mAb 10H8 (variable light and heavy chains of SEQ ID NOs: 24 and 30, respectively, with a murine IgG1 isotype) produced by hybridoma subclone_clone 10H8.25.6.10H8 or an antigen binding fragment of mAb 10H8.

In another embodiment, the isolated antibody or antigen binding fragment thereof binding to the epitope of SEQ ID NO: 11 (TREPK) is of any class of immunoglobulin, e.g., an IgA, IgD, IgE, IgG, and IgM, and several of these may be further divided into subclasses (isotypes), e.g., IgG-1, IgG-2, IgG-3 and IgG-4; IgA-1 and IgA-2. In a particularly useful embodiment, mAb 10H8 has a murine IgG1 isotype.

In another embodiment, the isolated antibody or antigen binding fragment thereof binding to the epitope of SEQ ID NO: 11 (TREPK) binds with a K_{d} value in the low micromolar (10⁻⁶) to nanomolar (10⁻⁷ to 10⁻⁹) range. In an embodiment, mAb 10H8 binds to h-Tau with a K_{d} of about 17 nM (see Example 3).

In another aspect, an isolated antibody or antigen binding fragment thereof is provided which specifically binds an epitope on h-Tau consisting of amino acids 189 to 194 (PKSGDR) as set forth in SEQ ID NO: 12.

In an embodiment, the isolated antibody or antigen binding fragment specifically binding to the epitope of SEQ ID NO: 12 (PKSGDR) comprises three light chain CDRs of SEQ ID NO: 32 (CDRL1), SEQ ID NO: 33 (CDRL2) and SEQ ID NO: 34 (CDRL3) and three heavy chain CDRs of SEQ ID NO: 38 (CDRH1), SEQ ID NO: 39 (CDRH2) and SEQ ID NO: 40 (CDRH3) or a variant of the antibody. In one embodiment, the variant of the antibody comprises 1, 2, 3, 4, 5, and 6 amino acid substitutions in one or more of the above recited CDRs, but retains the ability to bind an epitope of h-Tau consisting of amino acids 220 to 224 of SEQ ID NO: 11).

In another embodiment, the isolated antibody or antigen binding fragment thereof binding to the epitope of SEQ ID NO: 12 (PKSGDR) comprises a light chain variable region of SEQ ID NO: 36 and a heavy chain variable region of SEQ ID NO: 42 or a variant of the antibody. In one embodiment, the variant of the antibody comprises 1-20 amino acid substitutions in one or both of these sequences, but retains the ability to bind an epitope of h-Tau consisting of amino acids 189 to 194 (SEQ ID NO: 12).

In another embodiment, the isolated antibody or antigen binding fragment thereof binding to the epitope of SEQ ID NO: 12 (PKSGDR) is a mAb or antigen binding fragment thereof. In a particularly useful embodiment, the mAb is mAb 19G10 (variable light and heavy chains of SEQ ID NOs: 36 and 42, respectively, with a murine IgG2b isotype) produced by hybridoma subclone_clone 19G10.10.5.19G10 or an antigen binding fragment of mAb 19G10.

In another embodiment, the isolated antibody or antigen binding fragment thereof binding to the epitope of SEQ ID NO: 12 (PKSGDR) may be of any class of immunoglobulin, e.g., an IgA, IgD, IgE, IgG, and IgM, and several of these may be further divided into subclasses (isotypes), e.g., IgG-1, IgG-2, IgG-3 and IgG-4; IgA-1 and IgA-2.

In a particularly useful embodiment, the isolated antibody or antigen binding fragment thereof binding to the epitope of SEQ ID NO: 12 (PKSGDR), e.g., mAb 19G10 has an IgG2b isotype.

In another embodiment, the isolated antibody or antigen binding fragment thereof binding to the epitope of SEQ ID NO: 12 (PKSGDR) binds with a K_{d} value in the low micromolar (10⁻⁶) to nanomolar (10⁻⁷ to 10⁻⁹) range. In a further embodiment, mAb 19G10 binds to h-Tau with a K_{d} of about 6.3 nM (see Example 3).

### Nucleic Acid Molecules, Vectors and Host Cells

In another aspect, isolated nucleic acids are provided which encode the variable light and heavy chains of an antibody or antigen binding fragment thereof that specifically bind an epitope on h-Tau consisting of amino acids 220 to 224 (TREPK, SEQ ID NO: 11). In one embodiment, an isolated nucleic acid is provided which encodes one or both of an antibody light chain variable region and an antibody heavy chain variable region, wherein the antibody light chain variable region is of SEQ ID NO: 24 and an antibody heavy chain variable region is of SEQ ID NO: 30.

In another aspect, isolated nucleic acids are provided which encode the variable light and heavy chains of an antibody or antigen binding fragment thereof that specifically bind an epitope on h-Tau consisting of amino acids 189 to 194 (PKSGDR, SEQ ID NO: 12). In one embodiment, an isolated nucleic acid is provided which encodes one or both of an antibody light chain variable region and an antibody heavy chain variable region, wherein the antibody light chain variable region is of SEQ ID NO: 36 and an antibody heavy chain variable region is of SEQ ID NO: 42.

In another aspect, expression vectors are provided which comprise the isolated nucleic acids of the invention, wherein the nucleic acid is operably linked to control sequences that are recognized by a host cell when the host cell is transfected with the vector. Accordingly, in one embodiment, an expression vector is provided comprising one or both of the isolated nucleic acids of SEQ ID NO: 25 and SEQ ID NO: 31, or one or both of the isolated nucleic acids of SEQ ID NO: 37 and SEQ ID NO: 43.

Also provided are host cells comprising an expression vector and methods for producing the antibody or antigen binding fragment thereof disclosed herein comprising culturing a host cell harboring an expression vector encoding the antibody or antigen binding fragment in culture medium, and isolating the antigen or antigen binding fragment thereof from the host cell or culture medium.

### Tau 166 Peptide

In another aspect, an isolated peptide of SEQ ID NO: 9 known as Tau 166 peptide is provided, which is employed as an immunogen to make the antibodies of the present invention. Tau 166 peptide can be produced using standard recombinant methods. For example, an isolated nucleic acid encoding the Tau 166 peptide may be cloned into a suitable expression vector. In an embodiment, the isolated nucleic acid encoding Tau 166 peptide is SEQ ID NO: 10. The recombinant vector is then introduced into any suitable host cell. In one embodiment, the host cell is a sf9 (insect) cell. In another embodiment, the host cell is E. coli (see Example 1). Tau 166 peptide expressed from the host cell can then be purified from the host cell by standard methods (see e.g., Ausubel et al. (1991) Current Protocols in Molecular Biology Ch. 16 (John Wiley & Sons, NY).

### Methods of Making Antibodies and Antigen Binding Fragments Thereof

To produce antibodies, a suitable animal, such as a mouse, rat, hamster, monkey, or other mammal, is immunized with the Tau 166 peptide to produce antibody-secreting cells. In an embodiment, the animal, e.g., mouse, is immunized with Tau 166 peptide and an adjuvant which is used to enhance the immunological response. Examples of adjuvants include, but are not limited to, Freund's adjuvant (complete and incomplete), mineral salts such as aluminum hydroxide or aluminum phosphate, surface active substances, chitosan, lysolecithin, pluronic polyols, polyanions, peptides, oil emulsions (see Example 1 for immunization protocol). In another embodiment, the immune response to Tau 166 peptide may be enhanced by coupling the Tau 166 peptide to another immunogenic molecule or “carrier protein.” Examples of carrier proteins include, but are not limited to, keyhole limpet hemocyanin (KLH), tetanus toxoid, diphtheria toxoid, ovalbumin, cholera toxoid, and immunogenic fragments thereof. For guidance in coupling peptide immunogens to carrier proteins, see, e.g., Ausubel et al. (1989) Current Protocols in Molecular Biology Ch. 11.15 (John Wiley & Sons, NY); and Harlow and Lane (1988) Antibodies: A Laboratory Manual Ch. 5 (Cold Spring Harbor Laboratory, Cold Spring Harbor, N.Y.).

Hybridoma cells that produce parental (e.g. rodent) anti-h-Tau mAbs of the present invention may be produced by methods which are commonly known in the art. These methods include, but are not limited to, the hybridoma technique originally developed by Kohler, et al., (1975) (Nature 256:495-497), as well as the trioma technique (Hering, et al., (1988) Biomed. Biochim Acta. 47:211-216 and Hagiwara, et al., (1993) Hum. Antibod. Hybridomas 4:15), the human B-cell hybridoma technique (Kozbor, et al., (1983) Immunology Today 4:72 and Cote, et al., (1983) Proc. Natl. Acad. Sci. U.S.A 80:2026-2030), the EBV-hybridoma technique (Cole, et al., in Monoclonal Antibodies and Cancer Therapy, Alan R. Liss, Inc., pp. 77-96, 1985), and electric field based electrofusion using a Cyto Pulse large chamber cull fusion electroporator (Cyto Pulse Sciences, Inc., Glen Burnie, Md.). Preferably, mouse splenocytes are isolated and fused with PEG or by electrofusion to a mouse myeloma cell line based upon standard protocols. The resulting hybridomas may then be screened for the production of antigen-specific antibodies. For example, single cell suspensions of splenic lymphocytes from mice immunized with the Tau 166 antigen may be fused to SP2/0 nonsecreting mouse myeloma cells using e.g., a 50% polyethylene glycol-1500 (PEG-1500) solution in buffer, pH 8.0. Fused cells may be then plated onto microtiter plates and incubated in a hybridoma culture medium supplemented with HAT (liquid mixture of: sodium-hypoxanthine, aminopterin, and thymidine) for about two weeks. The culture supernatant from each individual plate may then be screened to identify antibody-secreting hybridomas by well-known methods such as enzyme-linked immunosorbent assay (ELISA). The antibody secreting hybridomas may be replated and screened again. If the screened hybridoma is still positive for the desired anti-h-Tau anatibodies, it can be subcloned at least twice. Subcloning can be carried out by limiting dilution, wherein the hybridoma cells are diluted in a culture medium by serial dilution to a final concentration of cells, e.g., 2.5 cells/mL. An aliquot of the cells, e.g., 200 μL (about ½ cell per well) is plated into each well and incubated from about two weeks. Single hybridoma cells in each well may then be microscopically identified and the supernatant from that single hybridoma may be screened by ELISA for the anti-h-Tau antibody of the present invention. Desired subclones are selected and can be expanded for antibody production or frozen in a liquid nitrogen freezer. When needed for studies, a vial of the frozen hybridoma may be thawed and grown in hybridoma culture medium to produce antibodies which can be purified. The procedure for making the anti-h-Tau antibodies of the present invention is described in Example 1.

The anti-h-Tau antibodies of the present invention may also be produced recombinantly (e.g., in an E. coli/T7 expression system). In this embodiment, nucleic acids encoding the antibody molecules of the invention (e.g., V_{H} or V_{L}) may be inserted into a pET-based plasmid and expressed in the E. coli/T7 system. There are several methods by which to produce recombinant antibodies which are known in the art. One example of a method for recombinant production of antibodies is disclosed in U.S. Pat. No. 4,816,567. Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455.

Anti-h-Tau antibodies can also be synthesized by any of the methods set forth in U.S. Pat. No. 6,331,415.

Mammalian cell lines available as hosts for expression of the antibodies or fragments disclosed herein are well known in the art and include many immortalized cell lines available from the American Type Culture Collection (ATCC). These include, inter alio, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Cell lines of particular preference are selected through determining which cell lines have high expression levels. Other cell lines that may be used are insect cell lines, such as Sf9 cells, amphibian cells, bacterial cells, plant cells and fungal cells. When recombinant expression vectors encoding the heavy chain or antigen binding portion or fragment thereof, the light chain and/or antigen binding fragment thereof are introduced into mammalian host cells, the antibodies are produced by culturing the host cells for a period of time sufficient to allow for expression of the antibody in the host cells or, more preferably, secretion of the antibody into the culture medium in which the host cells are grown.

Antibodies can be recovered from the culture medium using standard protein purification methods. Further, expression of antibodies of the invention (or other moieties therefrom) from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4.

### Diagnostic Assays, Methods of Treatment and Kits

In another aspect, a method of quantitating h-Tau in a biological sample is provided, the method comprising:
(a) contacting the biological sample with an anti-h-Tau antibody of the present invention, e.g., mAb 10H8 or mAb 19G10 or a variant of the antibody as described above, or an antigen binding fragment thereof under conditions allowing formation of an immune complex between h-Tau and the antibody or antigen binding fragment thereof, and
(b) detecting the immune complex formed.
   The aforementioned method can be used to quantify h-Tau in a biological sample as defined above, e.g., CSF, plasma, whole blood, serum or extracts of brain tissue.

In an embodiment of the aforementioned method for quantifying h-Tau, an anti-h-Tau of the present invention, e.g., mAb 10H8 or mAb 19G10 or a variant of the antibody, is coated onto a solid phase and the biological sample is then contacted with the solid phase. Examples of solid phases that may be used in this method are microtiter wells, plastic tubes, membranes, latex particles, magnetic particles, microspheres, and beads. The h-Tau in the biological sample binds to the antibody, and the amount of h-Tau can be determined by a direct or indirect method.

The direct method comprises detecting the presence of the h-Tau/anti-h-Tau antibody complex itself and thus the presence and amount of h-Tau by attaching a label to the antibody or antigen binding fragment thereof. Examples of labels are radioisotopes (such as ¹⁴C, ³⁵S, I¹²⁵ and ³H), enzymes having detectable reaction products (e.g., luciferase, beta-galactosidase, etc.), fluorescent labels (e.g., rhodamine, phycoerythrin, fluorescein isothiocyanate, resorufin etc.), chemiluminescent compounds (e.g., acridinium salts, luminol, isoluminol, etc.) and bioluminescent compounds (e.g., luciferin, aequorin, etc.).

In the indirect method, the anti-h-Tau antibody of the present invention can be labeled indirectly by reacting the anti-h-Tau antibody with a substance having affinity for the mouse anti-h-Tau antibody (e.g., goat anti-mouse or rabbit anti-mouse IgG) or a second antibody that has been labeled with a detectable reagent that is radioactive, fluorescent or chemiluminescent as mentioned above, and detecting the presence of the second antibody.

In another embodiment, the amount of h-Tau is quantified using a pair of anti-h-Tau antibodies, each specific for the conserved region of h-Tau spanning amino acids 104 to 277 of h-Tau. One of the pair of antibodies is an anti-h-Tau antibody of the present invention, e.g., mAb 10H8 or mAb 19G10 or a variant of the two antibodies, and the other antibody making up the pair is also specific for h-Tau. Examples of well-known anti-h-Tau antibodies, in particular mAbs that bind to an epitope on the conserved region of h-tau spanning amino acids 104 to 277 are Tau 5, BT2 and HT7 (commercially available at Covance). One of the pair of antibodies may be used as a “capture” antibody and the other of the pair may be used as a “detectably labeled antibody”. Accordingly, an embodiment uses a double antibody sandwich method for detecting h-Tau in a biological sample, wherein h-Tau is sandwiched between the capture antibody, i.e. mAb 10H8 or mAb 19G10 or a variant of the antibody, and the detectably labeled antibody, i.e., BT2, and wherein the capture antibody is immobilized onto a solid phase.

An embodiment of the double sandwich method is an enzyme-linked immunosorbent assay (ELISA) incorporating the use of an anti-h-Tau antibody or antigen binding fragment thereof of the present invention. For example, the ELISA comprises the following steps:

(a) coat a solid phase (e.g., surface of a microtiter plate well) with the anti-h-Tau antibody or antigen-binding fragment thereof of the present invention, e.g., mAb 10H8 or mAb 19G10 or a variant of these antibodies;

(b) apply a sample to be tested for the presence of h-Tau to the solid phase;

(c) wash the plate, so that unbound material in the sample is removed;

(d) apply a detectably labeled anti-h-Tau antibodies (e.g., enzyme-linked antibody) which is also specific to the h-Tau antigen, e.g., Tau 5, BT2 or HT7;

(e) wash the solid phase, so that the unbound, labeled antibody is removed;

(f) if the labeled antibody is enzyme-linked, apply a chemical which is converted by the enzyme into a fluorescent signal; and

(g) detect the presence of the labeled antibody.

As an example of the ELISA, the detectably labeled antibody is labeled with peroxidase which react with ABTS (e.g., 2,2′-azino-bis(3-ethylbenzthiazoline-6-sulphonic acid)) or 3,3′,5,5′-Tetramethylbenzidine to produce a color change which is detectable.

In a particularly useful embodiment of the double antibody sandwich assay, the amount of h-Tau in a sample of CSF is quantified utilizing a pair of anti-h-Tau antibodies of the present invention, e.g., mAb 10H8 and 19G10 or a variant of these antibodies, each of which specifically bind to non-identical epitopes on the conserved region of h-Tau spanning amino acids 104 to 277. The antibody that specifically binds the epitope of h-Tau consisting of amino acids 220 to 224 (TREPK, SEQ ID NO: 11), e.g., mAb 10H8 or a variant of the antibody, is used as the “capture antibody”, and the antibody that specifically binds the epitope of h-Tau consisting of amino acids 188 to 194 (PKSGDR, SEQ ID NO: 12), e.g., 19G10 or a variant of the antibody, is used as the “detectably labeled antibody”. This method for quantitating h-Tau in a CSF sample comprises:
(a) capturing h-Tau from the sample by contacting the sample with an antibody specifically binding to the epitope of h-Tau consisting of amino acids 220 to 224 (TREPK, SEQ ID NO: 11) e.g., 10H8 or a variant of the antibody, or an antigen binding fragment thereof under conditions allowing formation of a capture antibody/h-Tau complex, wherein the antibody or antigen binding fragment thereof is immobilized onto a solid support; and
(b) detecting the captured h-Tau by contacting the capture antibody/h-Tau complex with a detectably labeled antibody specifically binding to the epitope of h-Tau consisting of amino acids 189 to 194 (PKSGDR, SEQ ID NO: 12), e.g., 19G10 or a variant of the antibody, or an antigen binding fragment thereof under conditions allowing formation of a capture antibody/h-Tau/detectably labeled antibody complex.

As mentioned above, examples of solid supports are microtiter wells, plastic tubes, membranes, latex particles, magnetic particles, magnetic microparticles, microspheres, and beads. Suitable materials for the solid support include but are not limited to nylon, nitrocellulose, polyacrylamide, cellulose acetate, polystyrene, polypropylene, polymethacrylate, styrene, carboxylated styrene, and fiber-containing paper such as filter paper, chromatographic paper and glass fiber paper.

In a particularly useful embodiment, the solid support is magnetic microspheres (MagPlex® Microspheres which are carboxylated polystyrene micro-particles or beads, commercially available from Luminex Corporation, Austin, Tex.) (see e.g., U.S. Pat. Nos. 7,718,262, 6,514,295, 6,599,331, 6,632,526, 6,929,859, 7,445,844, 8,283,037 and 8,568,881). Reagents for labeling the “detectably labeled antibody” include but are not limited to a radioactive isotope, an enzyme, a biotin, dye, fluorescent label and chemiluminescent label. In a particular useful embodiment the reagent is biotin which is attached to a streptavidin-phyco erythrin conjugate.

An example of the aforementioned method for quantifying h-Tau in a biological sample, e.g., CSF, employs a bead-based technology (Luminex Corporation, Austin, Tex.), in which mAb 10H8 (the “capture antibody”), is coupled onto magnetic microspheres. The coupled microspheres are incubated with different concentrations of h-Tau (used as a standard) or CSF samples, together in the wells of a 96-well plate, followed by addition of biotinylated mAb 19G10 (the “detectably labeled antibody”) to form a mAb10H8/h-Tau/biotinylated mAb 19G10 complex. Detection of the biotinylated complex is carried out by incubation with a streptavidin-phycoerytherin conjugate which binds to the biotinylated antibody. An xMAP Technology instrument (FlexMap 3D, Luminex Corporation, Austin, Tex.) uses a classification laser (638 nM) to identify the specific microspheres, and a second reporter laser (532 nM) to excite the phycoerytherin molecule bound to conjugate. The fluorescent output from the phycoerythrin bound to the complex is measured using a detector (565 nM-585 nM) or CCD (Charged Coupled Device) imaging detector and is directly related to the amount of h-Tau in the CSF samples as read off a calibration curve prepared from the different concentration of h-Tau in the CSF sample.

In another aspect, a method for diagnosing AD in a patient suspected of having this disease is provided. This method can be used e.g., to select patients for treatment with an AD therapeutic agent, e.g., a BACE-1 inhibitor. The method comprises
(a) quantifying the amount of h-Tau in a biological sample obtained from the patient by:
   (i) capturing h-Tau from the sample by contacting the sample with an antibody specifically binding to the epitope of h-Tau consisting of amino acids 220 to 224 (TREPK, SEQ ID NO: 11) e.g., 10H8 or a variant of the antibody, or an antigen binding fragment thereof under conditions allowing formation of a capture antibody/h-Tau complex, wherein the antibody or antigen binding fragment thereof is immobilized onto a solid support; and
   (ii) detecting the captured h-Tau by contacting the capture antibody/h-Tau complex with a detectably labeled antibody specifically binding to the epitope of h-Tau consisting of 189 to 194 (PKSGDR, SEQ ID NO: 12), e.g., 19G10 or a variant of the antibody, or an antigen binding fragment thereof under conditions allowing formation of a capture antibody/Tau/detectably labeled antibody complex; and
(c) determining the concentration of h-Tau in the sample obtained in step (a), wherein a value greater than 184 pg/mL indicates a diagnosis of AD in the patient.

The method of diagnosing AD in a patient suspected of having AD by quantifying the amount of h-Tau is described, e.g., in Examples 4 and 5.

In an embodiment, the foregoing method for diagnosing Alzheimer's disease in a patient suspected of having this disease comprises:
(a) quantifying the amount of human Tau in a cerebrospinal fluid sample of the patient by:
(1) capturing human Tau from the sample by contacting the sample with an antibody or antigen binding fragment thereof, specifically binding to the epitope consisting of amino acids 220-224 of h-Tau, selected from the group consisting of:
   (i) an antibody or antigen binding fragment thereof comprising three light chain CDRs of SEQ ID NO: 20 (CDRL1), SEQ ID NO: 21 (CDRL2) and SEQ ID NO: 22 (CDRL3) and three heavy chain CDRs of SEQ ID NO: 26 (CDRH1), SEQ ID NO: 27 (CDRH2) and SEQ ID NO: 28 (CDRH3) or a variant of the antibody, and
   (ii) an isolated antibody or antigen binding fragment thereof comprising a light chain variable region of SEQ ID NO: 24 and a heavy chain variable region of SEQ ID NO: 30 or a variant of the antibody, under conditions allowing formation of a capture antibody/Tau complex, wherein the antibody or antigen binding fragment is immobilized onto a solid support; and
(2) detecting the captured Tau by contacting the capture antibody/Tau complex with a detectably labeled antibody or antibody fragment, specifically binding to the epitope consisting of amino acids 189 to 194, selected from the group consisting of:
   (i) an antibody or antigen binding fragment thereof comprising three light chain CDRs of SEQ ID NO: 32 (CDRL1), SEQ ID NO: 33 (CDRL2) and SEQ ID NO: 34 (CDRL3) and three heavy chain CDRs of SEQ ID NO: 38 (CDRH1), SEQ ID NO: 39 (CDRH2) and SEQ ID NO: 40 (CDRH3) or a variant of the antibody, and
   (ii) an antibody or antigen binding fragment thereof comprising a light chain variable domain of SEQ ID NO: 36 and a heavy chain variable domain of SEQ ID NO: 42 or a variant of the antibody, under conditions allowing formation of a capture antibody/Tau/detectable labeled antibody complex; and
(b) determining the concentration of human Tau in step (a), wherein a value greater than 184 pg/mL indicates a diagnosis of AD in the patient.
   In another embodiment of the foregoing methods for diagnosing AD by quantifying the amount of h-Tau, the method further comprises the steps of
(c) quantifying the amount of Aβ₁₋₄₂ in the CSF sample of the patient; and
(d) determining the ratio of h-Tau/Aβ₁₋₄₂ in the sample of the patient, wherein a ratio value greater than 0.215 indicates a diagnosis of AD in the patient.
   The amount of Aβ₁₋₄₂ in the CSF sample can be quantified in step (c) utilizing commercially available antibodies that specifically bind to an epitope on either the N-terminal and/or C-terminal ends of Aβ₁₋₄₂ in immunoassays as described above for quantifying h-Tau. Examples of commercially available antibodies include, but are not limited to, mAb 6E10 (N-terminal end, Covance), mAb 12F4 (C-terminal end, Covance), mAb 1-11-3 C-terminal end, BioLegend®), mAb G2-11(C-terminal end, EMD Millipore), and mAb 4G8 (N-terminal end, BioLegend®).
   In an embodiment, step (c) of the foregoing method of quantifying the amount of Aβ₁₋₄₂ in CSF comprises:
   capturing Aβ₁₋₄₂ from the sample by contacting the sample with an antibody or antigen binding fragment thereof specifically binding to an epitope on the C-terminal end of Aβ₁₋₄₂ under conditions allowing formation of a capture antibody/Aβ₁₋₄₂ complex, wherein the antibody or antigen binding fragment thereof is immobilized onto a solid support; and
   (ii) detecting the captured Aβ₁₋₄₂ by contacting the capture antibody/Aβ₁₋₄₂ complex with a detectably labeled antibody or antigen binding fragment thereof specifically binding to an epitope on the N-terminal end of Aβ₁₋₄₂ under conditions allowing formation of a detectably labeled antibody/Aβ₁₋₄₂/capture antibody complex.

As an example, Aβ₁₋₄₂ in CSF can be measured in step (c) using a sandwich ELISA system wherein a commercially available mAb such as mAb 6E10 is used as the capture antibody and alkaline phosphatase (AP)-conjugated mAb 12F4 as the detectably labeled antibody. In this system, a 96-well plate may be coated with mAb 6E10 by incubating overnight, and the plate then washed with buffer to remove unbound mAb 6E10. Diluted samples and a standard AB₁₋₄₂ peptide at varying concentration may then be incubated with AP-conjugated detectably labeled antibody, followed by addition of CDP-Star® Chemiluminescent Substrate (Applied Biosystems). The chemiluminescence may be measured with an EnVision® plate reader (Perkin Elmer).

In a particularly useful embodiment, the method for quantifying Aβ₁₋₄₂ in CSF in step (c) employs a bead-based technology (Luminex Corporation, Austin, Tex.), in which mAb 1-11-3 (BioLegend0), used as the “capture antibody” is coupled onto magnetic microspheres. The coupled microspheres are incubated with different concentrations of Aβ₁₋₄₂ (used as a standard) or CSF samples, together in the wells of a 96-well plate, followed by addition of biotinylated mAb 6E10 (the “detectably labeled antibody”) to form a mAb 1-11-3/Aβ₁₋₄₂/biotinylated mAb 6E10 complex (step (b) (ii)). Detection of the biotinylated complex is carried out by incubation with a streptavidin-phycoerytherin conjugate which binds to the biotinylated antibody. An xMAP technology instrument (FlexMap 3D, Luminex Corporation, Austin, Tex.) uses a classification laser (638 nM) to identify the specific microspheres, and a reporter laser (532 nM) to excite the phycoerytherin molecule bound to conjugate. The fluorescent output from the phycoerytherin bound to the complex is measured using a detector (565-585 nM) or CCD (Charged Coupled Device) imaging detector and is directly related to the amount of Aβ₁₋₄₂ in the CSF samples as read off a calibration curve prepared from the different concentration of Aβ₁₋₄₂ in the CSF sample.

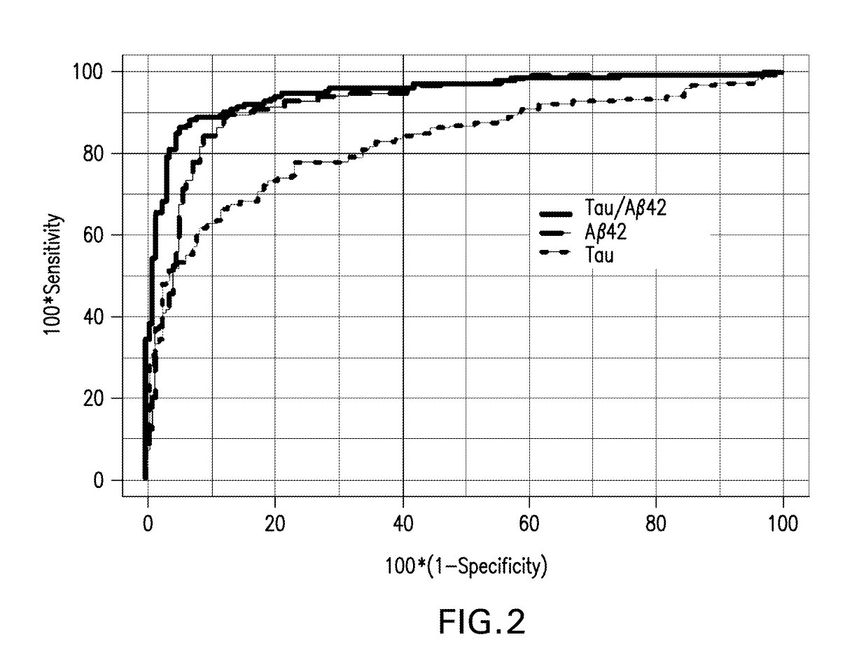

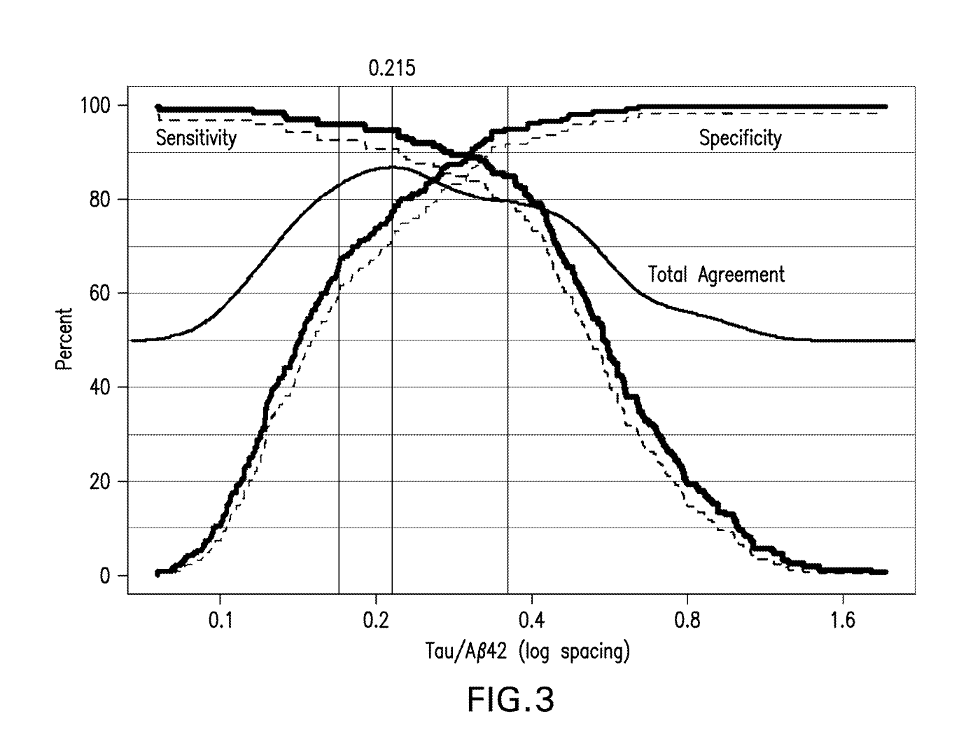

Following quantification of the amount of h-Tau and Aβ₁₋₄₂ in the CSF of the patient, the CSF h-Tau/Aβ₁₋₄₂ ratio of the patient is determined ((step (d)). As mentioned above, recent studies have shown that the ratio of CSF h-Tau/Aβ₁₋₄₂ is useful in identifying individuals with amyloid plaque pathology (Fagan et al., Arch. Neurol., Vol. 68, pp. 1137-1144, 2011). The CSF h-Tau/Aβ₁₋₄₂ ratio has also been shown to predict future cognitive decline in non-demented older adults and adults having mild AD (Fagan et al., Arch. Neurol., Vol. 64, pp. 343-349, 2007). Accordingly, the CSF h-Tau/Aβ₁₋₄₂ ratio can be used to as an aid in selecting patients for treatment with an AD modifying agent and was determined as set forth in Example 6 (See also Example 5 and FIGS. 2 and 3).

In another aspect, a method of treating AD in a patient in need thereof is provided, the method comprising:
(a) selecting a patient in need of treatment using the aforementioned diagnostic methods of quantifying h-Tau and/or the h-Tau/AP142 ratio; and
(b) administering to the patient a therapeutically effective amount of an AD therapeutic agent.

In an embodiment, the AD therapeutic agent is selected from those described above.

In one embodiment, the AD therapeutic agent is a BACE-1 inhibitor. BACE-1 has become an accepted therapeutic target for the treatment of Alzheimer's disease. For example, McConlogue et al., J. Biol. Chem., Vol. 282, No. 36 (September 2007), have shown that partial reductions of BACE-1 enzyme activity and concomitant reductions of Aβ levels lead to a dramatic inhibition of Aβ-driven AD-like pathology, making β-secretase a target for therapeutic intervention in AD. Ohno et al. Neurobiology of Disease, No. 26 (2007), 134-145, report that genetic deletion of BACE-1 in 5×FAD mice abrogates Aβ generation, blocks amyloid deposition, prevents neuron loss found in the cerebral cortex and subiculum (brain regions manifesting the most severe amyloidosis in 5×FAD mice), and rescues memory deficits in 5×FAD mice. The group also reports that Aβ is ultimately responsible for neuron death in AD and concludes that BACE-1 inhibition has been validated as an approach for the treatment of AD. Roberds et al., Human Mol. Genetics, 2001, Vol. 10, No. 12, 1317-1324, established that inhibition or loss of β-secretase activity produces no profound phenotypic defects while inducing a concomitant reduction in A13. Luo et al., Nature Neuroscience, Vol. 4, No. 3, March 2001, report that mice deficient in BACE-1 have normal phenotype and abolished β-amyloid generation.

In an embodiment of the aforementioned method for treating AD, the BACE-1 inhibitor is a compound described in WO2011044181, e.g., a compound selected from the group consisting of

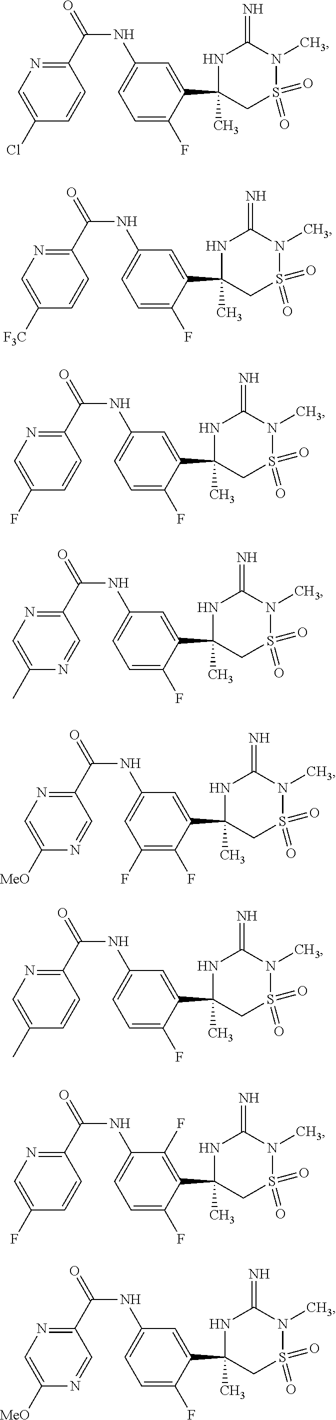

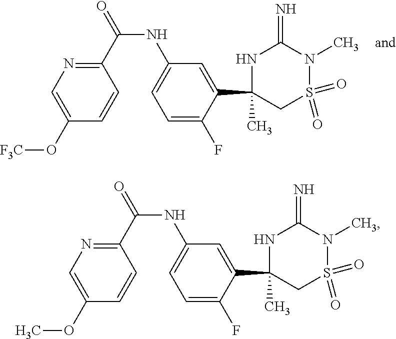 [SMILES: CN1C(=N)N[C@](C)(c2cc(NC(=O)c3ccc(OC(F)(F)F)cn3)ccc2F)CS1(=O)=O ; COc1ccc(C(=O)Nc2ccc(F)c([C@]3(C)CS(=O)(=O)N(C)C(=N)N3)c2)nc1]

or a tautomer, or the pharmaceutically acceptable salt of the compound or the tautomer.

In another embodiment, the BACE-1 inhibitor is verubecestat, which has the structure

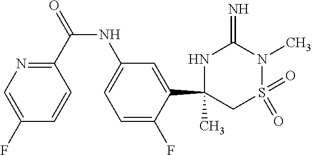 [SMILES: CN1C(=N)N[C@](C)(c2cc(NC(=O)c3ccc(F)cn3)ccc2F)CS1(=O)=O]

or a tautomer thereof. Pharmaceutically acceptable salts of verubecestat are also contemplated. Suitable acceptable salts include, but are not limited to, the HCl and the tosylate salts.

In another embodiment, the BACE-1 inhibitor is a compound described in WO2008/103351, e.g., a compound selected from the group consisting of

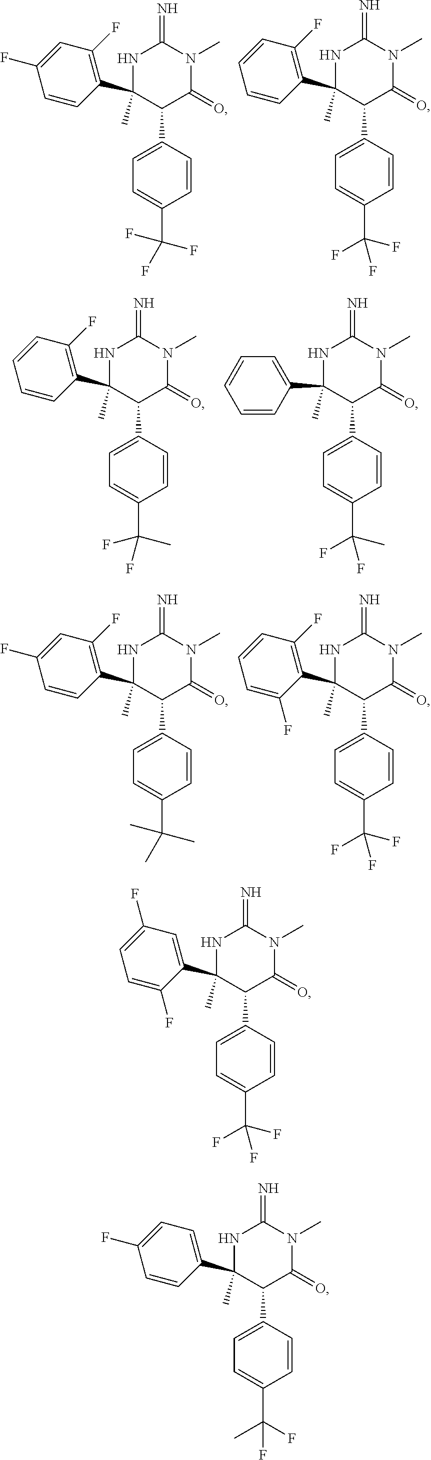

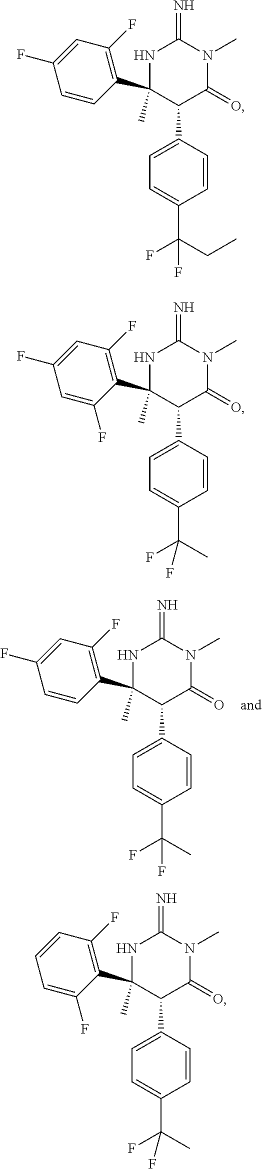

or a

a tautomer thereof, or a pharmaceutically acceptable salt of the compound or the tautomer.

It is also possible that the compounds referred to above having the moiety

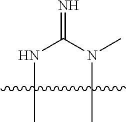

may exist in different tautomeric forms. All such forms are embraced within the scope of these BACE-1 inhibitors. Thus, for example, the BACE-1 inhibitors conforming to the formula:

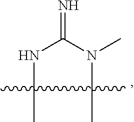

and their tautomers:

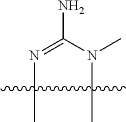

are both contemplated as being within the scope of the BACE-1 inhibitors described above.

Suitable doses for administering the aforementioned AD therapeutic agent such as a BACE-1 inhibitor to patients may readily be determined by those skilled in the art, e.g., by an attending physician, pharmacist, or other skilled worker, and may vary according to patient health, age, weight, frequency of administration, use with other active ingredients, and/or indication for which the compounds are administered. Doses may range from about 0.001 to 500 mg/kg of body weight/day of the AD therapeutic agent. In one embodiment, the dosage is from about 0.01 to about 25 mg/kg of body weight/day of the AD therapeutic agent. In another embodiment, the quantity of AD therapeutic agent in a unit dose of preparation may be varied or adjusted from about 1 mg to about 100 mg, preferably from about 1 mg to about 50 mg, more preferably from about 1 mg to about 25 mg, according to the particular application. In another embodiment, a typical recommended daily dosage regimen for oral administration can range from about 1 mg/day to about 500 mg/day, preferably 1 mg/day to 200 mg/day, in two to four divided doses.

In another embodiment, the AD therapeutic agent is the BACE-1 inhibitor having the structure

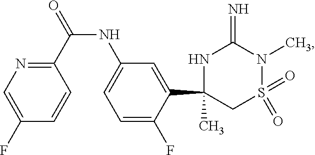 [SMILES: CN1C(=N)N[C@](C)(c2cc(NC(=O)c3ccc(F)cn3)ccc2F)CS1(=O)=O]

a tautomer thereof, or a pharmaceutically acceptable salt of the compound or the tautomer, and optionally pharmaceutically acceptable excipients suitable for formulation, wherein the dose is 5 mg, 10 mg, 12 mg, 40 mg, 60 mg or 100 mg per dose, given from 1 to 4 times per day. In a useful embodiment, the dose of this specific BACE-1 inhibitor is 40 mg or 60 mg once per day.

As discussed above, the amount and frequency of administration of the aforementioned AD therapeutic agent will be regulated according to the judgment of the attending clinician considering such factors as age, condition and size of the patient as well as severity of the symptoms being treated.

In an embodiment in which the AD therapeutic agent is a BACE-1 inhibitor, the BACE-1 inhibitor can be used in combination with another AD therapeutic agent. When used in combination with one or more additional AD therapeutic agents, the BACE-1 inhibitor may be administered together or sequentially. When administered sequentially, the BACE-1 inhibitor may be administered before or after the one or more additional AD therapeutic agents, as determined by those skilled in the art.

If formulated as a fixed dose, such combination products employ the BACE-1 inhibitor within the dosage range described herein and the other pharmaceutically active agent or treatment within its dosage range.

Accordingly, in an aspect, this invention includes combinations comprising an amount of at least one BACE-1 inhibitor, or tautomer, or a pharmaceutically acceptable salt of the BACE-1 inhibitor or tautomer, and an effective amount of one or more additional AD therapeutic agents described above.

The pharmacological properties of the aforementioned BACE-1 inhibitors may be confirmed by a number of pharmacological assays as exemplified in WO2011/044181.

For preparing pharmaceutical compositions from the aforementioned AD therapeutic agents, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example water or water-propylene glycol solutions may be used for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The aforementioned AD therapeutic agents may also be deliverable transdermally. The transdermal compositions can take the form of creams, lotions, aerosols and/or emulsions and can be included in a transdermal patch of the matrix or reservoir type as are conventional in the art for this purpose.

The aforementioned AD therapeutic agents may also be delivered subcutaneously.

In one embodiment, the AD therapeutic agent, e.g., a BACE-1 inhibitor, is administered orally.

In some embodiments, it may be advantageous for the pharmaceutical preparation comprising one or more AD therapeutic agents be prepared in a unit dosage form. In such forms, the preparation is subdivided into suitably sized unit doses containing appropriate quantities of the active component, e.g., an effective amount to achieve the desired purpose.

In another aspect, a kit is provided to quantify the amount of h-Tau in a biological sample for purposes such as diagnosing Alzheimer's disease and to select patients for AD treatment as described above. The kit comprises:
(a) an isolated antibody or antigen-binding fragment thereof specifically binding an epitope on h-Tau consisting of amino acids 220 to 224, e.g., mAb 10H8 or variant of the antibody as described above, or antigen binding fragment thereof; and
(b) an isolated antibody or antigen binding fragment specifically binding an epitope on h-Tau consisting of amino acids 189 to 194, e.g., mAb 19G10 or a variant of the antibody as described above, or antigen binding fragment thereof.

In an embodiment of the kit, the isolated antibody or antigen-binding fragment thereof of component (a) of the kit, e.g., mAb 10H8, is linked to a solid support as described above (e.g., magnetic microspheres), and component (b) of the kit, e.g., mAb 19G10, is biotinylated.

In another embodiment, the aforementioned kit may be used in conjunction with a second kit which includes a pair of antibodies specific for the Aβ₁₋₄₂ peptide as described above. In a further embodiment of the second kit, the antibodies specific for Aβ₁₋₄₂ are mAb 1-11-3 conjugated to magnetic microspheres, and biotinylated mAb 6E10.

The aforementioned kits can further include instructions for using the antibodies for a particular purpose, e.g., diagnosing AD patients for the purpose of selecting patients for treatment with an AD therapeutic agent, e.g, a BACE-1 inhibitor. The kit may also include buffers and other reagents that are routinely employed in a particular application, and substances for detecting labels, e.g., enzymatic substrates for enzyme labels, secondary labels such as a second antibody.

### EXAMPLES

### Example 1

### Preparation of the Monoclonal Antibodies 10H8 and 19G10

1. Preparation of Tau 166 Antigen

Tau 166 peptide (antigen) was expressed in E. coli (BL21(DE3)pLysS) by inoculating colonies from a recent transformation into LB (Luria-Bertani) culture medium containing 100 ug/mL ampicillin and 34 ug/mL chloramphenicol. After inoculation, the culture was grown to saturation overnight at 37° C. The overnight culture was used to inoculate 6×2 L at an initial optical density of 0.07 (A₆₀₀). The culture was incubated at 37° C. with shaking at 225 RPM to an optical density of 0.6 (A₆₀₀). Protein expression was induced through the addition of IPTG (Isopropyl-β-d-thiogalactopyranoside) to a final concentration of 1 mM at the same temperature. The culture was harvested via centrifugation at 9180×g for 10 minutes at 4 hours post-induction (A₆₀₀=0.63).

The harvested cell paste was suspended in 500 mL of lysis buffer (TBS-Tris Buffered Saline), pH 8.0 plus protease inhibitors). The resulting solution was briefly homogenized, and lysed via three passes through a microfluidizer. Lysates were clarified via centrifugation at 20,000×g for 20 minutes.

Tau 166 peptide was purified from the lysate using Ni-NTA (NTA-nitrilotriacetic acid) chromatography on a 2.5 cm Econo-column with a bed volume of 15 mL. Ni-NTA his-bind columns (Novagen) were pre-equilibrated in Buffer A (10 mM imidazole, pH 8.0). Tau 166 peptide in the soluble lysate fraction was batch bound to 15 mL of Ni-NTA resin at 4° C. for 2 hours. The resin was collected in the 2.5 cm Econo-column and washed in the following order; 10 column volumes of Buffer A, 10 column volumes of Buffer B (10 mM imidazole, 0.5% triton X-100, pH 8.0), 10 column volumes of Buffer C (10 mM imidazole, 0.5% Na-deoxycholate, pH 8.0), and finally 10 column volumes of Buffer A. Tau 166 peptide was eluted using a step gradient as follows: 10 column volumes of Buffer D (25 mM imidazole, pH 8.0), 10 column volumes of Buffer E (300 mM imidazole, pH 8.0). 5 mL fractions were collected and analyzed by SDS-PAGE prior to pooling elution fractions containing the protein of interest and the Buffer D wash.

The Ni-NTA pool was injected onto a 2.6 cm×60 cm SEC column (Superdex 200, GE Healthcare) (pre-equilibrated with Buffer (PBS, pH 7.4). Five mL fractions were collected over 1.1 column volumes and analyzed by SDS-PAGE prior to pooling fractions containing the protein of interest. The protein from this pool was concentrated two-fold using a 3000 kDa MWCO (Molecular weight cut-off) centrifugal device (PALL Corporation). Final stocks of Tau 166 peptide resuspended in PBS, pH 7.4 were analyzed for concentration and aliquots frozen at −80° C.

2. Immunization Protocol

Animals were immunized in preparation for use in the hybridoma fusion using a 38 day scheduled protocol. Briefly, the mice were injected on day 1 of the protocol using 50 μg of Tau 166 antigen in a complete Freund's adjuvant. Then, 28 days later the mice were injected again with 50 μg of antigen in an Incomplete Freund's adjuvant. 10 days later the animals were bled and the immunological responsiveness of the animal was determined through measurement of the EC50 dilution titer of the serum to the screening antigen, i.e., Tau 166 peptide. An animal with a titer value >50,000 was selected to be used in the fusion protocol. The animal was boosted with 50 μg of antigen for 3 consecutive days and then the animal was sacrificed on the fourth day for use in the fusion protocol.

3. Fusion Protocol

The hybridoma fusion procedure utilized SP2/0 mouse myeloma cell line as the fusion partner for the selected animal splenocytes. The SP2/0 were used in their log phase of growth and are >90% viable at time of fusion. The mouse spleen was harvested from the selected mouse and was perfused, macerated and strained. The cells were collected and counted. The SP2/0 were counted and an adequate amount was collected to allow for a fusion ratio of SP2/0 to splenocytes of 1:5 to 1:2.

Both cell preparations were washed twice in DMEM/F12 separately, then were combined and washed a third time. The supernatant was decanted and the resulting pellet was gently resuspended in the residual media. 1 ml of warmed PEG (polyethylene glycol) was added drop wise to the pellet over a 1 min period followed by 1 min of rest. A total of 10 mL of media was then added to the suspension over the next 3 minutes and the suspension was incubated for 5 minutes in a 37° C. water bath. The cells were spun down and resuspended in fusion media containing 20% Fetalclone and 2×HAT (liquid mixture of: sodium-hypoxanthine, aminopterin, and thymidine). The cells were then plated onto 96 well culture plates and incubated at 37° C. After 7 days an additional 105 μl of media containing 20% Fetalclone and 1×HAT was added to the cultures and the plates were incubated for an additional 7 days. At this point 80% of the media was removed from the wells and replaced with fresh media. The plates were incubated for an additional 7 days and then the supernatant from each well was screened by ELISA for antibody reactivity to the screening antigen(s).

4. ELISA Screening

For the screening of antigen, biotinylated Tau 166 peptide was bound to the surface of streptavidin coated 96 well culture plates and the wells were blocked with 150 μl of 1% BSA. Screening was performed by incubating 50 μl of 1% BSA block and 50 μl of culture supernatant from the fusion plates on the antigen coated and blocked plates. Detection was done using a goat anti-mouse IgG-HRP (horseradish peroxidase) conjugate and an ABTS (2,2′-Azinobis[3-ethylbenzothiazoline-6-sulfonic acid]) water soluble HRP substrate. Culture wells were considered ‘positive’ for antibody production if they resulted in an absorbance of greater than 0.5. The cells in these positive wells were harvested from the 96 well plates and were plated into 0.5 ml of media containing 20% Fetalclone and HT in 24 well culture plates. The positive wells were grown for 7 days and then were expanded to a second well with an additional 0.5 ml of HT media and were grown for an additional 3 days. The wells were screened against the screening antigen by ELISA, using both goat anti-mouse IgG-HRP and goat anti-mouse IgM-HRP to confirm that selected antibodies were IgG. Wells that were positive to only the IgG antibody with absorbance above 0.5 were then considered for sub-cloning.

Nine mAbs were developed using Tau 166 as the antigen and six of these tested as eight different pairs. All these pairs were tested for their diagnostic performance by measuring h-Tau concentration in the CSF of AD patients and NEV. ROC curves and their corresponding AUC (area under the curve) values were calculated. The antibody pair with the highest AUC value was found to be 10H8 and 19G10, indicating their superiority in distinguishing normal subjects from AD subjects over the rest of the pairs tested. Thus, these two mAbs were selected for further development.

5. Subcloning

The cells from wells screened as positive to mouse IgG expanded into 2 wells of a 24 well plate were incubated until they were 50% confluent and greater than 90% viable. The cells were pooled and counted. Enough cells were then removed to create a suspension containing 40 ml of 5 cell/ml in 20% Fetalclone media. The remaining cells were frozen back. The cells were plated at 105 μl/well on 3×96 well plates at the equivalent of 0.5 cell per well. The plates were incubated for approximately a week, then were fed with an additional 105 μl of media and were grown for another week or until they were >25% confluent. The wells containing single “colonies” of growing cells were selected for screening by ELISA with goat anti-mouse IgG-HRP (horseradish peroxidase) as described previously. The process was repeated using wells that were positive to the screening antigen until greater than 90% of the screened clones were positive to the Tau 166 antigen. At this point a subset of the positive clones were expanded into 1 ml of media in a 12 well culture plate. The cells were expanded into multiple wells and frozen back. A cell bank was produced from a selection of these clones.

6. GMP (Good Manufacturing Practice) Cell Bank Production

The GMP cell bank was grown from the frozen back subclone. A small volume of culture was grown in 10% Fetalclone media to produce samples for use in pre-banking quality screening. This screening includes bioburden sterility testing and mycoplasma detection testing. Cells were frozen back for the QC testing and for the generation of the cell bank after test results were received.

An aliquot of the cells that have passed quality testing were grown up and bulked to an appropriate volume for banking vials at 5×10⁶ c/ml. The culture was isotyped prior to banking. The cells were counted every 24-72 hours and expanded by dilution to 5×10⁴ c/ml in media. When an adequate volume of culture had been produced, cells were counted and if the viability was greater than 91%, the banking proceeded. Cell culture was spun down and the supernatant was discarded. Cells were resuspended in the appropriate amount of cell freezing media (90% Fetalclone, 10% DMSO). The cells were immediately placed in cryotubes, placed at −70° C. for >24-72 hours, and then were stored in liquid nitrogen. After at least 24 hours in liquid nitrogen, post banking QC was performed. Representative cell vials from the beginning, middle and end of the fill process were thawed and grown. Doubling time of the culture from the beginning and end of the fill were performed. Isotyping of the culture grown from the mid-fill samples was performed and the culture was harvested and sent for post bank mycoplasma testing, and samples were sent for bioburden sterility. The bank was released when all quality results were in and negative.

7. 10H8 and 19G10 Antibody Production

Antibody production was done from the released GMP cell bank. A vial of banked cells was thawed and cultured in 10% Fetalclone media. Culture was counted every 24-72 hours and expanded as necessary by diluting the culture to 5×10⁴ cells/ml. The culture was expanded to the appropriate volume and counted and if the viability was determined to be greater than 30%, the supernatant was harvested. The culture was spun at 3000×g for 20 minutes and the supernatant was decanted into a proper storage vessel. The supernatant was isotyped using the IsoStrip Mouse Monoclonal Isotyping Kit (Roche Applied Science, Indianapolis, Ind.) at this stage, and then stored at 2-8° C. until purification.

8. Purification

Supernatant grown from culture from the GMP cell bank was purified using the AKTA liquid chromatography system on a dedicated protein A column. The antibody was bound to the column using a pH 8.8 buffer and eluted using a pH 3.0 buffer. The product (10H8 or 19G10) was buffer dialyzed and concentrated and the final buffer was PBS, pH 7.4. The product (10H8 or 19G10) was tested and was greater than 90% pure by HPLC and protein concentration was measured by A280 (absorbance assay measuring protein concentration at 280 nM). Product was stored in 500 μs aliquots at 2.0 mg/ml and stored at −10° C. to −25° C. mAb 10H8 has variable light and heavy chain sequences of SEQ ID NOs: 24 and 30, respectively, with a murine IgG1 isotype. mAb 19G10 has variable light and heavy chain sequences of SEQ ID NOs: 36 and 42, respectively, with a muring IgG2b isotype.

### Example 2

### Epitope Mapping of 10H8 and 19G10

Epitope mapping of clones 10H8 and 19G10 was completed using JPT Peptide Technologies' RepliTope™ peptide microarrays. This technology consists in using purified synthetic peptides chemoselectively and covalently immobilized to the glass surface. An optimized hydrophilic linker moiety is inserted between the glass surface and the antigen derived peptide sequence to avoid false negatives caused by sterical hindrance. The peptides used spanned Tau mid region protein sequence of 166 amino acids between amino acid 104 and amino acid 269 (Tau 166). Peptides of 15 amino acids in length that extended downstream either 1 or 2 amino acids at a time were used in this experiment.

The assay was performed using an automated TECAN HS4X00 microarray processing station. Microarrays were washed, incubated with blocking buffer for 60 min at 30° C., and subsequently with clones 101-18 and 19G10 diluted in blocking buffer for 120 min at 30° C. Microarrays were washed and incubated with secondary antibody diluted in blocking buffer for 45 min at 30° C. and then dried. The quantification was performed using high resolution fluorescence scanner. The resulting images were analyzed and quantified using spot-recognition software GenePix (Molecular Devices). For each spot, the mean signal intensity was extracted and expressed as arbitraty florescence units.

The results for the epitope mapping of 10H8 and 19G10 are shown in Tables 4 and 5. The first column shows the peptides containing putative epitopes used in the RepliTope™ microarray experiment, whereas the second column shows the arbitrary fluorescence units from that experiment. Bolded numbers of arbitrary fluorescence units are indicative of strong reactivity, and thus presence of the epitope. Based on this reactivity, it was concluded that the epitope for mAb 10H8 consists of amino acids TREPK and for mAb 19G10 the epitope consists of amino acids PKSGDR.

TABLE 4

| ID | Fluorescent Intensity |
|---|---|
| PGSRSRTPSLPTPPT | 570.33 |
| (SEQ ID NO: 44) | |
| SRSRTPSLPTPPTRE | 552.00 |
| (SEQ ID NO: 45) | |
| SRTPSLPTPPTREPK | 57972.33 |
| (SEQ ID NO: 46) | |
| TPSLPTPPTREPKKV | 56714.33 |
| (SEQ ID NO: 47) | |
| SLPTPPTREPKKVAV | 63929.00 |
| (SEQ ID NO: 48) | |
| PTPPTREPKKVAVVR | 53033.33 |
| (SEQ ID NO: 49) | |
| PPTREPKKVAVVRTP | 61139.33 |
| (SEQ ID NO: 50) | |
| TREPKKVAVVRTPPK | 32352.00 |
| (SEQ ID NO: 51) | |
| EPKKVAVVRTPPKSP | 2101.67 |
| (SEQ ID NO: 52) | |
| KKVAVVRTPPKSPSS | 750.33 |
| (SEQ ID NO: 53) | |
| SLPTPPTREPKKVAV | 64393.00 |
| (SEQ ID NO: 54) | |
| LPTPPTREPKKVAVV | 59474.00 |
| (SEQ ID NO: 55) | |
| PTPPTREPKKVAVVR | 64238.67 |
| (SEQ ID NO: 56) | |
| TPPTREPKKVAVVRT | 60638.00 |
| (SEQ ID NO: 57) | |
| PPTREPKKVAVVRTP | 60153.33 |
| (SEQ ID NO: 58) | |
| PTREPKKVAVVRTPP | 64284.33 |
| (SEQ ID NO: 59) | |
| TREPKKVAVVRTPPK | 58813.67 |
| (SEQ ID NO: 60) | |
| REPKKVAVVRTPPKS | 1399.33 |
| (SEQ ID NO: 61) | |
| EPKKVAVVRTPPKSP | 889.67 |
| (SEQ ID NO: 62) | |

### Epitope Mapping Result for Clone 10H8=TREPK

TABLE 5

| ID | Fluorescent Intensity |
|---|---|
| PPAPKTPPSSGEPPK | 891.67 |
| (SEQ ID NO: 63) | |
| APKTPPSSGEPPKSG | 760.67 |
| (SEQ ID NO: 64) | |
| KTPPSSGEPPKSGDR | 55751.67 |
| (SEQ ID NO: 65) | |
| PPSSGEPPKSGDRSG | 62047.67 |
| (SEQ ID NO: 66) | |
| SSGEPPKSGDRSGYS | 62721.33 |
| (SEQ ID NO: 67) | |
| GEPPKSGDRSGYSSP | 58008.00 |
| (SEQ ID NO: 68) | |
| PPKSGDRSGYSSPGS | 53418.00 |
| (SEQ ID NO: 69) | |
| KSGDRSGYSSPGSPG | 1814.00 |
| (SEQ ID NO: 70) | |
| GDRSGYSSPGSPGTP | 588.33 |
| (SEQ ID NO: 71) | |
| SSGEPPKSGDRSGYS | 55441.00 |
| (SEQ ID NO: 72) | |
| SGEPPKSGDRSGYSS | 59686.33 |
| (SEQ ID NO: 73) | |
| GEPPKSGDRSGYSSP | 58865.67 |
| (SEQ ID NO: 74) | |
| EPPKSGDRSGYSSPG | 63089.00 |
| (SEQ ID NO: 75) | |
| PPKSGDRSGYSSPGS | 58692.67 |
| (SEQ ID NO: 76) | |
| PKSGDRSGYSSPGSP | 49555.33 |
| (SEQ ID NO: 77) | |
| KSGDRSGYSSPGSPG | 1548.67 |
| (SEQ ID NO: 78) | |
| SGDRSGYSSPGSPGT | 618.67 |
| (SEQ ID NO: 79) | |

### Epitope Mapping Result for Clone 19G10=PKSGDR

### Example 3

### Measurement of Relative Binding Affinity of mAbs 10H8 and 19G10

The binding affinity (Kd) of the 10H8 and 19G10 mAbs was determined by BIAcore CM3 sensor chip (Biacore, Piscataway, N.J.) using immobilized h-Tau 441 as the capture protein.

TABLE 6

| Antibody | Kₒₙ (M⁻¹s⁻¹) | K_{off} (s⁻¹) | K_{d} (nM) |
|---|---|---|---|
| 10H8 mAb | 1.82E+04 ± 9.84E+03 | 3.06E−04 ± 1.50E−04 | 17 ± 2.1 |
| 19G10 mAb | 2.59E+04 ± 7.05E+03 | 1.76E−04 ± 1.37E−04 | 6.3 ± 3.5 |

### Example 4

### Quantification of hTau and A131-42

Preparation of h-Tau 441 Standard for h-Tau Assay

The sequence for h-Tau 441 (SEQ ID NO: 1) was cloned into the pet3A vector at the NDE I/BamH I cleavage site. The His-tag, TEV cleavage site, and the h-Tau 441 sequence are shown in Table 1. The vector was transformed into in E. coli (BL21 (DE3)pLysS) and protein expression induced through the addition of IPTG. Purification of h-Tau 441 was completed using Ni-NTA His-bind columns (Novagen).

h-Tau Assay

The h-Tau assay employed a bead-based technology (Luminex Corporation, Austin, Tex.), in which Tau specific mAb (mAb10118, Merck) was coupled onto magnetic microspheres at a ratio of 100 μg mAb 10118:1.0 mL MagPlex® microspheres, using a two-step carbodiimide reaction protocol. In the two-step procedure carboxyl groups on the surface of the microsphere are first activated with the carbodiimide derivative EDC (1-Ethyl-3-(3-dimethylaminopropyl)carbodiimide) to form an intermediate that is stabilized with sulfo-NHS (N-Hyroxysulfosuccinimide sodium salt). The intermediate then reacts with a protein's primary amide to form an amide bond, thus creating a stable conjugated protein on the surface of the microsphere. In the assay the coupled microspheres were incubated with calibrators (h-Tau 441: 15.6-1000 pg/mL) or CSF samples, together in the wells of a 96-well plate for 2 hours at room temperature with shaking. Biotinylated mAb specific to h-Tau (mAb 19G10, Merck), labeled at a 20-fold molar excess, were then added to the reaction and incubated for 1 hour at room temperature with shaking, followed by 30 minute incubation with streptavidin-phycoerythrin (SAPE) conjugate (Moss, Inc., Pasadena, Md.) which bound to the biotinylated antibody. Between each of the incubation steps 2×1541 wash (PBS-TBN) was employed using a magnetic wash system. After completion of the reactions the microspheres were re-suspended in 100 μL wash buffer and then analyzed immediately on a xMAP instrument (FlexMap 3D, Luminex Corporation, Austin, Tex.) that employs a classification laser (638 nm) or classification excitation (621 nm) to identify the specific microspheres, and a reporter laser (532 nm) or reporter excitation (511 nm) to excite the phycoerithrin molecule bound to the conjugate. The fluorescent output is directly related to the concentration of h-Tau in the samples as read off a prepared calibration curve.

Aβ₁₋₄₂ Assay

The Aβ₁₋₄₂ assay also employed a bead-based technology (Luminex Corporation, Austin, Tex.), in which mAb 1-11-3 (BioLegend) was coupled onto magnetic microspheres (MagPlex® microspheres) using a two-step carbodiimide reaction protocol. In the two-step procedure carboxyl groups on the surface of the microsphere are first activated with the carbodiimide derivative EDC (1-Ethyl-3-(3-dimethylaminopropyl)carbodiimide) to form an intermediate that is stabilized with sulfo-NHS (N-Hyroxysulfosuccinimide sodium salt). The intermediate then reacts with a protein's primary amide to form an amide bond, thus creating a stable conjugated protein on the surface of the microsphere. In the assay the coupled microspheres were incubated with calibrators (standard Aβ₁₋₄₂: 5.47-700 pg/mL) or CSF samples, together in the wells of a 96-well plate for 2 hours at room temperature with shaking. Biotinylated 6E10 mAb (Covance) labeled at a 20-fold molar excess, was then added to the reaction and incubated for 1 hour at room temperature with shaking, followed by 30 minute incubation with streptavidin-phycoerythrin (SAPE) conjugate (Moss, Inc., Pasadena, Md.) which binds to the biotinylated antibody. Between each of the incubation steps 2×150 μL wash (PBS-TBN) was employed using a magnetic wash system. After completion of the reactions the microspheres were re-suspended in 100 μL wash buffer and then analyzed immediately on an XMAP instrument that employs a classification laser (638 nm) or classification excitation (621 nm) to identify the specific microspheres, and a reporter laser (532 nm) or reporter excitation (511 nm) to excite the phycoerithrin molecule bound to conjugate. The fluorescent output is directly related to the concentration of Aβ₁₋₄₂ analyte in the samples as read off a prepared calibration.

### Example 5

### Correlation Between AD Diagnosis and h-Tau Levels and h-Tau/Aβ₄₂ Ratio

h-Tau levels in CSF of human individuals were determined in a set of representative healthy controls (n=188) and AD subjects (n=155) using the h-Tau assay described in Example 4. The CSF was collected per institutional guidelines. The healthy controls (HC) and AD patients were similar in gender (45% males for AD and 45% males in HC) and age (mean age 64 years old in AD, and 67 years old in healthy volunteers). As shown in Table 7 below, mean CSF h-Tau concentrations were higher in AD subjects (208±83) as compared with healthy control subjects (126±39). The mean ratio of h-Tau/Aβ₁₋₄₂ was 0.175±0.096 in healthy controls, whereas it was 0.613±0.302 in AD. This raw data represents the ability to use the h-Tau levels or ratio of h-Tau/Aβ₁₋₄₂ to distinguish subjects that are AD or HC.

TABLE 7

| | Clinical Dx | (N) | Mean | SD |
|---|---|---|---|---|
| Tau | Healthy Control | 188 | 126 | 39 |
| | AD | 155 | 208 | 83 |
| Tau/AB42 | Healthy Control | 188 | 0.175 | 0.096 |
| | AD | 155 | 0.613 | 0.302 |

### Example 6

### Method for Establishing Cut-Off Values for h-Tau and h-Tau/Aβ₁₋₄₂ Ratio

### Statistical Analysis Plan

CSF samples were collected from 188 HC and 155 AD subjects from five international sites and assayed using the h-Tau and Aβ₁₋₄₂ assays, described above. A two-step approach was used to establish the cut-off. First, a range of possible cut-off values which best differentiate AD vs. healthy controls was determined that distinguish AD from HC with at least 80% sensitivity and 60% specificity using the h-Tau/Aβ₁₋₄₂ ratio. Receiver-operator characteristic (ROC, see Pepe, M. S. The Statistical Evaluation of Medical Tests for Classification and Prediction. 2003 Oxford University Press: Oxford, Great Britain) curve methodology was used to characterize the performance of the assays in CSF to discriminate between samples from HC and AD subjects. Second, Positron Emission Tomography (PET) imaging using Vizamyl™ (¹⁸F-Flutemetamol) as approved for imaging of the brain to estimate A13 neuritic plaque presence in adult patients with cognitive impairment who are being evaluated for AD and other causes of cognitive decline (General Electric Vizamyl™ package insert, Revised October, 2013) was performed and results were used to select a specific cut-off value within the established range. Images were scored as either positive or negative scans following the recommended methods for image orientation and display of these brain regions as described in the FDA approved label for Vizamyl™. The healthy controls and AD subjects with amyloid PET imaging were used both to estimate sensitivity and specificity in the first step and estimate PET concordance in the second step. The CSF hTau and hTau/Aβ₁₋₄₂ value within the window that met our sensitivity and specificity criteria and maximized concordance with amyloid PET was 184 pg/mL for hTau and a ratio of 0.215 for hTau/Aβ₁₋₄₂ (FIGS. 2 and 3).

1. An isolated antibody or antigen binding fragment thereof that specifically binds an epitope on human Tau consisting of amino acids 220 to 224.

2. An isolated antibody or antigen binding fragment of claim 1, which comprises three light chain CDRs of SEQ ID NO: 20 (CDRL1), SEQ ID NO: 21 (CDRL2) and SEQ ID NO: 22 (CDRL3) and three heavy chain CDRs of SEQ ID NO: 26 (CDRH1), SEQ ID NO: 27 (CDRH2) and SEQ ID NO: 28 (CDRH3).

3. The isolated antibody or antigen binding fragment of claim 1, which comprises a light chain variable region of SEQ ID NO: 24 and a heavy chain variable region of SEQ ID NO: 30.

4. The isolated antibody or antigen binding fragment of claim 1, which is the monoclonal antibody 10H8 or antigen binding fragment thereof.

5. (canceled)

6. (canceled)

7. (canceled)

8. An isolated antibody or antigen binding fragment thereof that specifically binds an epitope on human Tau consisting of amino acids 189 to 194.

9. The isolated antibody or antigen binding fragment of claim 8, which comprises three light chain CDRs of SEQ ID NO: 32 (CDRL1), SEQ ID NO: 33 (CDRL2) and SEQ ID NO: 34 (CDRL3) and three heavy chain CDRs of SEQ ID NO: 38 (CDRH1), SEQ ID NO: 39 (CDRH2) and SEQ ID NO: 40 (CDRH3).

10. The isolated antibody or antigen binding fragment of claim 8, which comprises a light chain variable domain of SEQ ID NO: 36 and a heavy chain variable domain of SEQ ID NO: 42.

11. The isolated antibody or antigen binding fragment of claim 8, which is the monoclonal antibody 19G10 or antigen binding fragment thereof.

12. (canceled)

13. (canceled)

14. (canceled)

15. (canceled)

16. (canceled)

17. (canceled)

18. (canceled)

19. A method of quantitating human Tau in a biological sample, the method comprising:
(a) contacting the biological sample with an antibody or antigen binding fragment of claim 1 under conditions allowing formation of an immune complex between human Tau and the antibody or antigen binding fragment thereof; and
(b) detecting the immune complex formed.

20. The method of claim 19, wherein the antibody is monoclonal antibody 10H8 or an antigen binding fragment thereof.

21. A method of quantitating human Tau in a biological sample, the method comprising:
(a) contacting the biological sample with the antibody of antigen binding fragment of claim 8 under conditions allowing formation of an immune complex between human Tau and the antibody or antigen binding fragment thereof; and
(b) detecting the immune complex formed.

22. The method of claim 21, wherein the antibody is monoclonal antibody 19G10 or an antigen binding fragment thereof.

23. A method for quantitating human Tau in a cerebrospinal fluid sample, the method comprising:
(a) capturing human Tau from the sample by contacting the sample with the antibody or antigen binding fragment thereof of claim 1 under conditions allowing formation of a capture antibody/Tau complex, wherein the antibody or antigen binding fragment is immobilized onto a solid support; and
(b) detecting the captured Tau by contacting the capture antibody/Tau complex with a detectably labeled antibody or antibody fragment thereof of claim 8 under conditions allowing formation of a capture antibody/Tau/detectable labeled antibody complex.

24. The method of claim 23, wherein the capture antibody is monoclonal antibody 10H8 or antigen binding fragment thereof and the detectably labeled antibody is monoclonal antibody 19G10 or an antigen binding fragment thereof.

25. The method of claim 23, wherein the solid support is selected from the group consisting of magnetic particles, microspheres, magnetic microspheres, beads, membranes, plastic tubes, microtiter wells.

26. The method of claim 25, wherein the solid support is a magnetic microsphere.

27. The method of claim 23, wherein the detectably labeled antibody is labeled with a reagent selected from the group consisting of a radioactive isotope, an enzyme, a biotin, dye, fluorescent label and chemiluminescent label.

28. The method of claim 27, wherein the reagent is biotin.

29. The method of claim 28, wherein the biotin is attached to a streptavidin-phycoerythrin conjugate.

30. A method for diagnosing Alzheimer's disease in a patient suspected of having this disease, the method comprising:
(a) quantifying the amount of human Tau in a cerebrospinal fluid sample of the patient using the method of claim 23; and
(b) determining the concentration of human Tau in step (a), wherein a value greater than 184 pg/mL indicates a diagnosis of AD in the patient.

31. The method of claim 30, further comprising
(a) quantifying the amount of Aβ₁₋₄₂ in the cerebrospinal fluid sample of the patient; and
(b) determining the ratio of human Tau/Aβ₁₋₄₂ in the sample of the patient, wherein a ratio value greater than 0.215 indicates a diagnosis of AD in the patient.

32. The method of claim 31, wherein in step (c) the amount of Aβ₁₋₄₂ is quantified utilizing at least one monoclonal antibody selected from the group consisting of 6E10, 12F4, 1-11-3, G2-11 and 4G8, or an antigen binding fragment of any of these antibodies.

33. The method of claim 31, wherein in step (c) the amount of Aβ₁₋₄₂ in the cerebrospinal fluid sample is quantified by:
(i) capturing Aβ₁₋₄₂ from the sample by contacting the sample with an antibody or antigen binding fragment thereof specifically binding to an epitope on the C-terminal end of Aβ₁₋₄₂ under conditions allowing formation of a capture antibody/Aβ₁₋₄₂ complex, wherein the antibody or antigen binding fragment thereof is immobilized onto a solid support; and
(ii) detecting the captured Aβ₁₋₄₂ by contacting the capture antibody/Aβ₁₋₄₂ complex with a detectably labeled antibody or antigen binding fragment thereof specifically binding to an epitope on the N-terminal end of Aβ₁₋₄₂ under conditions allowing formation of a detectably labeled antibody/Aβ₁₋₄₂/capture antibody complex.

34. The method of claim 33, wherein the antibody used in step (c)(i) is monoclonal antibody 1-11-3 and the antibody used in step (c)(ii) is monoclonal antibody 6E10.

35. A method for treating Alzheimer's disease in a patient in need thereof, the method comprising:
(a) selecting a patient in need of treatment by
(i) quantifying the amount of human Tau in a cerebrospinal fluid sample of the patient using the method of claim 23; and
(ii) determining the concentration of human Tau in step (i), wherein a value greater than 184 pg/mL indicates a diagnosis of AD in the patient; and
(b) administering to the patient a therapeutically effective amount of an AD therapeutic agent.

36. The method of claim 35, wherein the AD therapeutic agent is a BACE-1 inhibitor.

37. The method of claim 36, wherein the BACE-1 inhibitor is a compound selected from the group consisting of 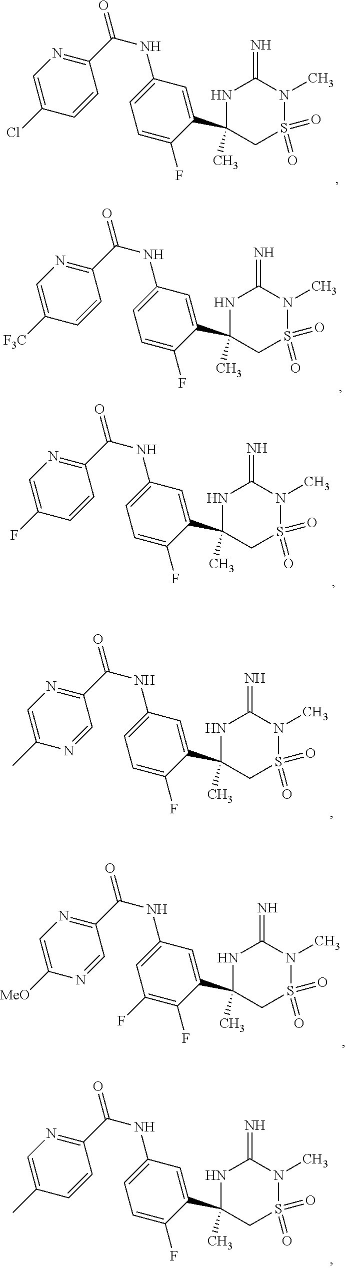 [SMILES: CN1C(=N)N[C@](C)(c2cc(NC(=O)c3ccc(Cl)cn3)ccc2F)CS1(=O)=O ; CN1C(=N)N[C@](C)(c2cc(NC(=O)c3ccc(C(F)(F)F)cn3)ccc2F)CS1(=O)=O ; CN1C(=N)N[C@](C)(c2cc(NC(=O)c3ccc(F)cn3)ccc2F)CS1(=O)=O ; Cc1cnc(C(=O)Nc2ccc(F)c([C@]3(C)CS(=O)(=O)N(C)C(=N)N3)c2)cn1 ; COc1cnc(C(=O)Nc2cc(F)c(F)c([C@]3(C)CS(=O)(=O)N(C)C(=N)N3)c2)cn1 ; Cc1ccc(C(=O)Nc2ccc(F)c([C@]3(C)CS(=O)(=O)N(C)C(=N)N3)c2)nc1] 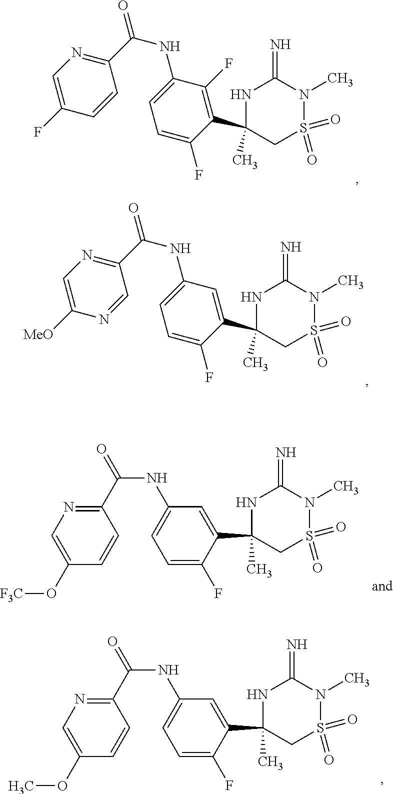 [SMILES: CN1C(=N)N[C@](C)(c2c(F)ccc(NC(=O)c3ccc(F)cn3)c2F)CS1(=O)=O ; COc1cnc(C(=O)Nc2ccc(F)c([C@]3(C)CS(=O)(=O)N(C)C(=N)N3)c2)cn1 ; CN1C(=N)N[C@](C)(c2cc(NC(=O)c3ccc(OC(F)(F)F)cn3)ccc2F)CS1(=O)=O ; COc1ccc(C(=O)Nc2ccc(F)c([C@]3(C)CS(=O)(=O)N(C)C(=N)N3)c2)nc1]
or a tautomer thereof, or pharmaceutically acceptable salt of the compound or tautomer.

38. The method of claim 37, wherein the BACE-1 inhibitor has the structure 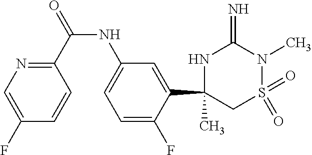 [SMILES: CN1C(=N)N[C@](C)(c2cc(NC(=O)c3ccc(F)cn3)ccc2F)CS1(=O)=O]
or a tautomer thereof, or pharmaceutically acceptable salt of the compound or the tautomer.

39. The method of claim 36, wherein the BACE-1 inhibitor is a compound selected from the group consisting of 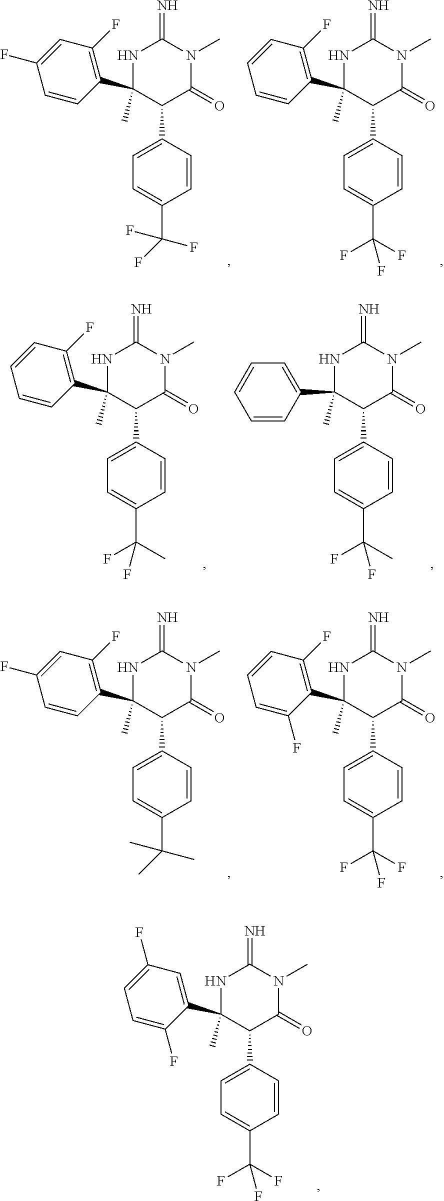 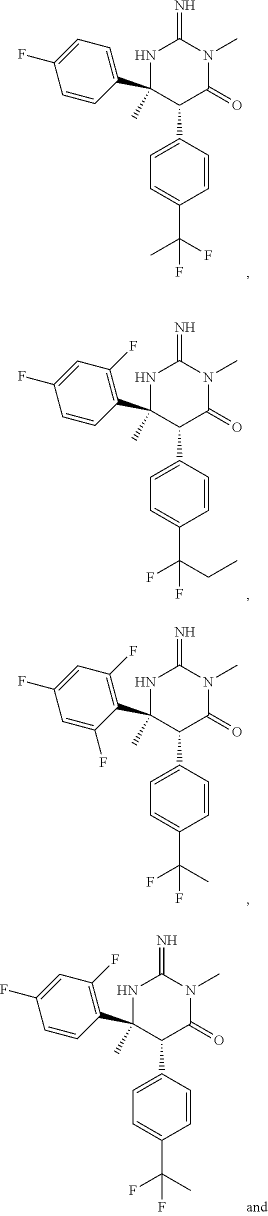 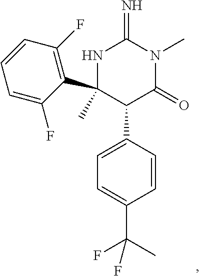 [SMILES: CN1C(=N)N[C@](C)(c2c(F)cccc2F)[C@@H](c2ccc(C(C)(F)F)cc2)C1=O]
a tautomer thereof, or a pharmaceutically acceptable salt of the compound or tautomer.

40. (canceled)

41. (canceled)
